(19) United States
(12) Patent Application Publication
(10) Pub. No.: US 2024/0252474 A1
(43) Pub. Date: Aug. 1, 2024

(54) METHODS AND COMPOSITIONS COMPRISING A CARDIO-TOLERATED HDAC INHIBITOR

(71) Applicant: HUYABIO International, LLC, San Diego, CA (US)

(72) Inventors: Gloria LEE, Santa Monica, CA (US); Mireille GILLINGS, San Diego, CA (US)

(21) Appl. No.: 18/421,171

(22) Filed: Jan. 24, 2024

Related U.S. Application Data
(60) Provisional application No. 63/441,131, filed on Jan. 25, 2023.

(51) Int. Cl. A61K 31/4406; A61K 39/00; A61K 39/395; A61P 35/00

(52) U.S. Cl. CPC A61K 31/4406; A61K 39/3955; A61P 35/00; A61K 2039/505; A61K 2039/545

(57) ABSTRACT

Provided herein are methods comprising an HDAC inhibitor (HDACi), and/or a PD-L1 and/or a PD-1 inhibitor, and/or a CTLA-4 inhibitor, and/or an anti-cancer agent. Also, provided herein are pharmaceutical compositions suitable for treatment of cancer.

### CROSS REFERENCE

This application claims the benefit of U.S. Provisional Application No. 63/441,131, filed Jan. 25, 2023, which application is incorporated herein by reference in its entirety.

### FIELD

The present invention relates to compounds comprising HDAC inhibitors and the use of such compounds. The present invention is also related to methods comprising HDAC inhibitors, PD-1 inhibitors, PD-L1 inhibitors, and CTLA-4 inhibitors amongst other checkpoint inhibitors, and the use thereof.

### BACKGROUND OF THE INVENTION

Many commonly used drugs are known to disrupt cardiac rhythm and cause prolongation of the QT interval that leads to fatal ventricular arrhythmias and sudden cardiac death. For example, drugs like antihistamines, antimicrobials, antidepressants, antibiotics, and anti-cancer drugs are known to have an effect on cardiac rhythm.

Cancer is a significant cause of morbidity and mortality worldwide for which standard of care has greatly improved over the years, including the use of known anti-cancer drugs comprising epigenetic modifiers, such as histone deacetylase inhibitors (HDACi) in combination with immuno-oncology agents targeting cytotoxic T-lymphocyte-associated protein 4 (CTLA-4), the programmed cell death receptor-1 (PD-1) and its ligand PD-L1. However, many cancer chemotherapeutic agents are known to prolong the QT interval or to increase the risk of prolonged QT interval. Predicting the risks involved with most drugs especially with anti-cancer drugs is difficult, since these drugs are often structurally and pharmacologically diverse. Accordingly, there is a need in the art for new anti-cancer therapies that cause zero to minimum disruption of the cardiac rhythm. Provided herein are solutions to these and other problems in the art.

### SUMMARY OF THE INVENTION

Provided herein, inter alia, are methods that include a compound comprising an HDAC inhibitor (HDACi). The method includes administering to a subject an effective amount of an HDACi such that the administration of the HDACi causes no increase in QTc, QTcF, or heart rate (HR). In some embodiments, the method further includes administration of a CTLA-4 inhibitor, PD-L1 inhibitor, and/or PD-1 inhibitors. In some embodiments, the CTLA-4 inhibitor, the PD-L1 inhibitor, and/or PD-1 inhibitors is an antibody. In some embodiments, the method is used to treat cancer.

In some embodiments of the disclosure provided herein is a method of treating a subject with a therapeutically effective amount of a HDACi that causes no increase in QTc, QTcF, or heart rate (HR), wherein the HDACi comprises a compound of formula I, or pharmaceutically acceptable salts, stereoisomers, prodrugs, enantiomers, diastereomers, hydrates, co-crystals, or polymorphs thereof, wherein formula I is:

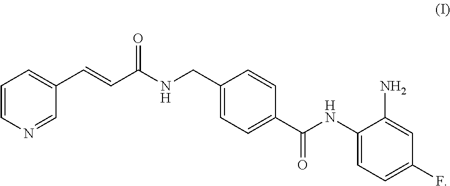 [SMILES: Nc1cc(F)ccc1NC(=O)c1ccc(CNC(=O)/C=C/c2cccnc2)cc1]

In some embodiments, said compound of formula I is N-(2-amino-4-fluorophenyl)-4-[[[(2E)-1-oxo-3-(3-pyridinyl)-2-propen-1-yl]amino]methyl]benzamide or a pharmaceutically acceptable salt or solvate thereof. In some embodiments, the method further comprising administering a CTLA-4 inhibitor, a PD-1 inhibitor or a PD-L1 inhibitor.

In some other embodiments of the present disclosure comprises a method for treating cancer, said method comprising administering a therapeutically effective amount of a HDACi to a subject, wherein administration of the HDACi to the subject causes no increase in QTc, QTcF, or heart rate (HR). In some embodiments, the method causes no increase in mean QTc, median QTc, mean QTcF, median QTcF, mean heart rate (HR) or median HR. In some embodiments, administering the HDACi to the subject causes a decrease in mean QTc, median QTc, mean QTcF, median QTcF, mean heart rate (HR) or median HR. In some embodiments, administering the HDACi to each subject at increasing dosage results in reduced mean QTcF or median QTcF. In some embodiments, administering the HDACi to each subject results in no change in mean HR or median HR, or causes a decrease in mean HR or median HR. In some embodiments, the method further comprises administering a CTLA-4 inhibitor, a PD-1 inhibitor or a PD-L1 inhibitor.

Some other embodiments of the present disclosure comprise a pharmaceutical composition comprising an effective amount of the HDACi, wherein the effective amount is effective to treat cancer and causes no increase in QTc, QTcF, or heart rate (HR).

In some embodiments, the HDACi inhibits class I and class IIb HDACs. In some embodiments, the HDACi inhibits one or more of HDAC1, HDAC2, HDAC3, or HDAC10. In some embodiments, the HDACi inhibits all of HDAC1, HDAC2, HDAC3, and HDAC10. In some embodiments, the HDACi is tucidinostat (chidamide/HBI-8000).

In some embodiments, described herein are combinations (e.g., combination therapies, such as therapeutic methods and uses, kits and compositions) for treating diseases comprising cancer. The said kit comprising a combination of any of one of the embodiments described herein or a pharmaceutical composition of the embodiments described herein. In some embodiments, the kit further comprises at least one administration device. In some embodiments, components in the kit are sterilized. In some embodiments, the combinations described herein include an HDAC inhibitor and an anti-cancer agent, a PD-L1 inhibitor, PD-1 inhibitor, and/or further a CTLA-4 inhibitor. In some embodiments, the combinations described herein include an HDAC inhibitor and an anti-cancer agent.

In some embodiments, the effective amount of the HDACi is an amount effective to treat cancer. In some embodiments, the cancer is an advanced solid tumor. In some embodiments, the solid tumor is cancer such as melanoma, renal cell carcinoma, or non-small cell lung cancer (NSCLC). In some embodiments, the cancer can be a hematological cancer such as lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CML), acute lymphocytic leukemia, (ALL), or chronic lymphocytic leukemia (CLL). In some embodiments, the cancer is Hodgkin's Lymphoma or Reed-Sternberg disease. In some embodiments, the cancer is relapsed or refractory peripheral T-cell lymphoma (“RR/PTCL”) or relapsed or refractory aggressive adult T-cell lymphoma (“RR/ATL”)

In some embodiments, the effective amount of the said compound is about 5 mg to about 80 mg per day. In some embodiments, the HDACi is administered as an oral dose. In some embodiments, the HDACi is administered at a dose of about 20 mg, about 30 mg, or about 40 mg once daily during a cycle. In some embodiments, the cycle is at least about two days in duration. In some embodiments, the cycle is from about one week to about four weeks in duration. In some embodiments, the method further comprises administering a PD-1 inhibitor or a PD-L1 inhibitor. In some embodiments, the PD-1 inhibitor or PD-L1 inhibitor is administered on day 2 of the cycle. In some embodiments, the PD-1 inhibitor is an anti-PD-1 antibody. In some embodiments, the anti-PD-1 antibody is nivolumab. In some embodiments, the anti-PD-1 antibody is pembrolizumab. In some embodiments, the PD-1 antibody is administered at a dose of 240 mg per administration every two (2) weeks. In some other embodiments, the anti-PD-1 antibody can be administered according to established regimens such as those provided in a package insert.

Some embodiments of the present disclosure comprise a first pharmaceutical composition and a second pharmaceutical composition. The first pharmaceutical composition comprises the said HDACi and the second pharmaceutical comprises the said anti-cancer agent, said PD-1 inhibitor, PD-L1 inhibitor and/or CTLA-4 inhibitor. In some embodiments, the first pharmaceutical composition is formulated for oral administration. In other embodiments, the second pharmaceutical composition is formulated for parenteral administration.

In some embodiments, said anti-cancer agent, said PD-1 inhibitor, PD-L1 inhibitor and/or CTLA-4 inhibitor is a small molecule compound, a nucleic acid, a peptide, a protein, an antibody, a peptibody, a diabody, a minibody, a single-chain variable fragment (ScFv), or a fragment or variant thereof. In some embodiments, at least one of the CTLA-4 inhibitor, PD-L1 inhibitor or the PD-1 inhibitor is an antibody. In some embodiments, said inhibitor antibody is a monoclonal antibody. In some embodiments, said inhibitor antibody comprises a human antibody, a mouse antibody, a chimeric antibody, a humanized antibody, or a chimeric humanized antibody. In some embodiments, said inhibitor antibody is a human antibody or a humanized antibody. In some embodiments, said inhibitor antibody is present at an amount of about 0.1 mg/kg to about 30 mg/kg. In some embodiments, said inhibitor antibody is present at an amount of about 0.5 mg/kg to about 15 mg/kg. In some embodiments, said inhibitor antibody is present at an amount of about: 0.1 mg/kg, 0.3 mg/kg, 1 mg/kg, 2 mg/kg, 2.5 mg/kg, 3 mg/kg, 5 mg/kg, 10 mg/kg, or 20 mg/kg. In some embodiments, said combination is suitable for parenteral administration to a cancer patient. In some embodiments, said parenteral administration comprises intravenous (IV) administration.

In other embodiments, the PD-L1 inhibitor is an antibody such as durvalumab, avelumab, atezolizumab, BMS-936559, STI-A1010, STI-A1011, STI-A1012, STI-A1013, STI-A1014, or STI-A1015 (Sorrento Therapeutics).

In other embodiments, the PD-1 inhibitor is an antibody such as nivolumab, pembrolizumab, pidilizumab, REGN2810 (also known as SAR-439684), PDR001, SHR-1210, or MEDI0680.

In still other embodiments, the CTLA-4 inhibitor is an antibody comprising ipilimumab.

In some embodiments, said cancer is a solid tumor cancer such as squamous cell carcinoma, nonsquamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral nerve sheath tumor (MPNST). In some embodiments, said cancer is non-small cell lung cancer (NSCLC), hepatocellular carcinoma, melanoma, ovarian cancer, breast cancer, pancreatic cancer, renal cell carcinoma (RCC), bladder cancer or colorectal cancer. In some embodiments, said cancer is lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CML), acute lymphocytic leukemia, (ALL), or chronic lymphocytic leukemia (CLL). In some embodiments, said cancer patient is treatment naïve. In some embodiments, said cancer patient is treatment naïve for non-small cell lung cancer (NSCLC), hepatocellular carcinoma, melanoma, ovarian cancer, breast cancer, pancreatic cancer, renal cell carcinoma, or colorectal cancer. In some embodiments, said combination is administered to the cancer patient as a first line therapy. In some embodiments, said combination is administered to the cancer patient as a second, third, fourth, fifth, or sixth line of treatment. In some embodiments, said combination is administered to said cancer patient following treatment with at least one anti-cancer therapy. In some embodiments, said anti-cancer therapy comprises chemotherapy, radiotherapy, surgery, targeted therapy, immunotherapy, or a combination thereof. In some embodiments, said cancer is resistant to at least one anti-cancer agent.

In some embodiments, said compound of formula I and said inhibitor of said combination are administered simultaneously or sequentially. In some embodiments, said compound of formula I is administered 2 to 3 times per week. In some embodiments, said compound of formula I is administered daily. In some embodiments, said PD-L1 inhibitor, PD-1 inhibitor, and/or CTLA-4 inhibitor, and said compound of formula I are concomitantly administered on day 1 of an administration regimen. In some embodiments, said combination is administered to said patient as a regimen. In some embodiments, said regimen is repeated until disease progression or unacceptable toxicity. In some embodiments, said regimen comprises a rest period of at least 1 day between consecutive administration periods. In some embodiments, said compound of formula I of said combination is administered 2 to 3 times per week in said regimen and said PD-L1 inhibitor, PD-1 inhibitor, and/or CTLA-4 inhibitor is administered every 2 to 3 weeks. In some embodiments, said compound of formula I of said combination is administered once a day (“QD”) for 21 days in said regimen and said inhibitor antibody is administered every 2 to 3 weeks.

In some embodiments, said method causes no increase in QTc, QTcF, or heart rate (HR). In some embodiments, said method causes a decrease in QTc, QTcF, or HR in the subject. In some embodiments, administration of HDACi to a subject in need thereof at increasing dosage results in reduced QTcF. In some embodiments, said method causes no change in HR or a decrease in HR.

In some embodiments, said method of treating cancer inhibits metastasis of said cancer in said patient. In some embodiments, said method of treating cancer reduces tumor or tumor burden in said patient. In some embodiments, said method of treating cancer inhibits pre-existing metastasis of said cancer in said patient. In some embodiments, said method of treating cancer prolongs the time to disease progression of said cancer in said patient. In some embodiments, said method of treating cancer prolongs the survival of said patient. In some embodiments, said method of treating cancer increases progression-free survival of said patient.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B show mean HBI-8000 plasma concentration amongst patient treatment groups including a combination of compounds of formula I, and a PD-1 inhibitory antibody. The dosing of each treatment is differentiated using different colors as seen in the figure legend.

FIG. 2 shows a plot of log QTcF versus log RR for Cycle 1 with patient regression lines and population regression line included.

FIG. 3 shows the change from baseline QTcF versus HBI-8000 concentration with population regression line and 90% confidence bands. The dosing of each treatment is differentiated using different colors as seen in the figure legend.

FIG. 4 shows waterfall plots of maximum changes of target lesions by tumor type.

FIG. 5 shows swimmer plots of tumor responses over the course of study treatment.

### DETAILED DESCRIPTION

### Definitions

All patents, applications, published applications and other publications cited herein are incorporated by reference in their entirety. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts. Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

“QT” interval is a measurement that represents the total time from ventricular depolarization to complete repolarization. This measurement begins at the start of the “Q” wave to the end of the “T” wave, where QT is expressed in milliseconds. “QT” interval as described herein is in accordance with its plain and ordinary meaning as understood in the art.

“QTc” indicates the corrected QT interval. A corrected QT interval estimates the QT at a heart rate of 60 bpm (beats per minute) to allow comparison of values over different heart rates.

“QTc” as described herein is in accordance with its plain and ordinary meaning as understood in the art.

“QTc” correction using Bazett's formula, QTc=QT/(RR)^{1/2}, where RR is the interval from one R peak to the next.

“QTc” correction using Fridericia: QT(Fri)=QT/(RR)^{1/3}, where RR is the interval from one R peak to the next.

“QTcF” indicates the QT interval corrected by Fridericia's cube root formula, QTcF=QT/(RR)^{1/3}. “QTcF” as described herein is in accordance with its plain and ordinary meaning as understood in the art.

“Heart rate” (or “HR”) indicates the number of heart beats per minute or 60/RR, wherein RR is the R to R interval expressed in seconds per beat.

The terms “P wave”, “Q wave”, “T wave”, “RR”, “QRS complex”, “PR interval”, “ST segment”, U wave as described herein are in accordance with their plain and ordinary meaning as understood in the art.

The term “PD-L1 inhibitor” refers to a moiety (e.g., compound, nucleic acid, polypeptide, antibody) that decreases, inhibits, blocks, abrogates or interferes with the activity, binding of PD-L1 to its receptor, PD-1, or expression of PD-L1 (e.g., Programmed Cell Death 1 Ligand; PD-L1 (CD274); GI: 30088843), including variants, isoforms, species homologs of human PD-L1 (e.g., mouse) and analogs that have at least one common epitope with PD-L1. A PD-L1 inhibitor includes molecules and macromolecules such as, for example, compounds (small molecule compounds), nucleic acids, polypeptides, antibodies, peptibodies, diabodies, minibodies, single-chain variable fragments (ScFv), and fragments or variants thereof. Thus, a PD-L1 inhibitor as used herein refers to any moiety that antagonizes PD-L1 activity, its binding to PD-1, or its expression. PD-L1 inhibitor efficacy can be measured, for example, by its inhibitor concentration at 50% (half-maximal inhibitor concentration or IC₅₀). PD-L1 inhibitors include exemplary compounds and compositions described herein. A PD-L1 inhibitor antibody refers to a PD-L1 inhibitor which is a monoclonal or polyclonal antibody as described herein.

The terms “durvalumab,” “avelumab,” “atezolizumab,” “BMS-936559,” “STI-A1010,” “STI-A1011,” “STI-A1012,” “STI-A1013,” “STI-A1014,” and “STI-A1015” are used in accordance with their plain and ordinary meaning as understood in the art.

The term “PD-1 inhibitor” refers to a moiety (e.g., compound, nucleic acid, polypeptide, antibody) that decreases, inhibits, blocks, abrogates or interferes with the activity or expression of PD-1 (e.g., Programmed Cell Death Protein 1; PD-1 (CD279); GI: 145559515), including variants, isoforms, species homologs of human PD-1 (e.g., mouse) and analogs that have at least one common epitope with PD-1. A PD-1 inhibitor includes molecules and macromolecules such as, for example, compounds, nucleic acids, polypeptides, antibodies, peptibodies, diabodies, minibodies, single-chain variable fragments (ScFv), and fragments or variants thereof. Thus, a PD-1 inhibitor as used herein refers to any moiety that antagonizes PD-1 activity or expression. PD-1 inhibitor efficacy can be measured, for example, by its inhibitor concentration at 50% (half-maximal inhibitor concentration or IC₅₀). PD-1 inhibitors include exemplary compounds and compositions described herein. A PD-1 antibody refers to a PD-1 inhibitor which is a monoclonal or polyclonal antibody as described herein.

The terms “nivolumab,” “pembrolizumab,” “pidilizumab,” “AMP-224,” “REGN2810,” “PDR 001,” “SHR-1210,” “SAR-439684” and “MEDI0680” are used in accordance with their plain and ordinary meaning as understood in the art.

The term “CTLA-4 inhibitor” refers to a moiety (e.g., compound, nucleic acid, polypeptide, antibody) that decreases, inhibits, blocks, abrogates or interferes with the activity or expression of CTLA-4, including variants, isoforms, species homologs of human CTLA-4 (e.g., mouse) and analogs that have at least one common epitope with CTLA-4. A CTLA-4 inhibitor includes molecules and macromolecules such as, for example, compounds, nucleic acids, polypeptides, antibodies, peptibodies, diabodies, minibodies, single-chain variable fragments (ScFv), and fragments or variants thereof. Thus, a CTLA-4 inhibitor as used herein refers to any moiety that antagonizes CTLA-4 activity or expression. CTLA-4 inhibitor efficacy can be measured, for example, by its inhibitor concentration at 50% (half-maximal inhibitor concentration or IC₅₀). CTLA-4 inhibitors include exemplary compounds and compositions described herein. A CTLA-4 antibody refers to a CTLA-4 inhibitor which is a monoclonal or polyclonal antibody as described herein.

The term “ipilimumab” is used in accordance with their plain and ordinary meaning as understood in the art.

The terms “polypeptide” and “protein” are used interchangeably herein and refer to any molecule that includes at least 2 or more amino acids.

The term “Inhibitor Antibody” refers to a monoclonal or polyclonal antibody that binds to its substrate or target with sufficient strength to inhibit activity of the substrate or target. As used herein, an “Inhibitor Antibody” comprises a PD-L1 inhibitor antibody, a PD-1 inhibitor antibody, and/or a CTLA-4 inhibitor antibody.

The term “effective amount” refers to the amount of a therapy (e.g., used in a method provided herein) which is sufficient to accomplish a stated purpose or otherwise achieve the effect for which it is administered. An “effective amount” can be sufficient to reduce and/or ameliorate the progression, development, recurrence, severity and/or duration of a given disease, disorder or condition and/or a symptom related thereto, or can be sufficient to reduce the level of activity or binding of a polypeptide (e.g., PD-L1, PD-1, CTLA-4). An “effective amount” can be a “therapeutically effective amount” which refers to an amount sufficient to provide a therapeutic benefit such as, for example, the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. A “therapeutically effective amount” used in a method described herein can enhance the therapeutic efficacy of another therapeutic agent.

The term “regimen” refers to a protocol for dosing and timing the administration of one or more therapies (e.g., methods described herein) for treating a disease, disorder, or condition described herein. A regimen can include periods of active administration and periods of rest as known in the art. Active administration periods include administration of combinations and compositions described herein and the duration of time of efficacy of such combinations and compositions. Rest periods of regimens described herein include a period of time in which no compound is actively administered, and in some instances, includes time periods where the efficacy of such compounds can be minimal. Combination of active administration and rest in regimens described herein can increase the efficacy and/or duration of administration of the combinations and compositions described herein.

The terms “therapies” and “therapy” refer to any protocol(s), method(s), and/or agent(s) that can be used in the prevention, treatment, management, and/or amelioration of a disease, disorder, or condition or one or more symptoms thereof. In some instances the term refers to active agents such as an anti-cancer agent described herein. The terms “therapy” and “therapy” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

The term “patient” or “subject” refers to a mammal, such as a human, bovine, rat, mouse, dog, monkey, ape, goat, sheep, cow, or deer. Generally a patient as described herein is human.

The terms “inhibition,” “inhibit,” “inhibiting” refer to a reduction in the activity, binding, or expression of a polypeptide or reduction or amelioration of a disease, disorder, or condition or a symptom thereof “Inhibiting” as used here can include partially or totally blocking stimulation, decreasing, preventing, or delaying activation or binding, or inactivating, desensitizing, or down-regulating protein or enzyme activity or binding.

Antibodies described herein can be polyclonal or monoclonal and include xenogeneic, allogeneic, or syngeneic forms and modified versions thereof (e.g., humanized or chimeric). An “antibody” is intended to mean a polypeptide product of lymphocytes within the immunoglobulin class of polypeptides that is able to bind to a specific molecular antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxy-terminal portion of each chain includes a constant region. (See Borrebaeck (ed.) (1995) Antibody Engineering, Second Edition, Oxford University Press; Kuby (1997) Immunology, Third Edition, W.H. Freeman and Company, New York). Specific molecular antigens that can be bound by an antibody described herein include PD-L1, PD-1, CTLA-4, and their epitopes.

The term “monoclonal antibody(ies)” refers to a population of antibody molecules that contain one species of an antigen binding site capable of immunoreacting with a particular epitope of an antigen, whereas the term “polyclonal antibody(ies)” refers to a population of antibody molecules that contain multiple species of antigen binding sites capable of interacting with a particular antigen. A monoclonal antibody, typically displays a single binding affinity for a particular antigen with which it immunoreacts. For example, the monoclonal antibodies to be used in accordance with the present invention can be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J Mol. Biol. 222: 581-597 (1992); Sidhu et al., J. Mal. Biol. 338(2): 299-310 (2004); Lee et al., J. Mal. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lon berg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein also include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, pp. 6851-6855 (1984)). “Humanized antibody(ies)” can be considered as a subset of chimeric antibodies described herein.

The term “human” when used in reference to an antibody or a functional fragment thereof (e.g., “humanized antibody(ies))” refers an antibody or functional fragment thereof that has a human variable region or a portion thereof corresponding to human germline immunoglobulin sequences. Such human germline immunoglobulin sequences are described by Kabat et al. (1991), Sequences of Proteins of Immunological Interest, Fifth Edition, U.S. Department of Health and Human Services, NIH Publication No. 91-3242. A human antibody, in the context of the present invention, can include an antibody that binds to PD-L1 or variants thereof as described herein.

In some instances a human antibody is an antibody that possesses an amino acid sequence corresponding to that of an antibody produced by a human and/or has been made using any of the techniques for making human antibodies as disclosed herein. Human antibodies can be produced using various techniques known in the art, including phage-display libraries. Hoogenboom and Winter, J. Mol. Biol., 227:381 (1991); Marks et al., J. Mal. Biol., 222:581 (1991). Also available for the preparation of human monoclonal antibodies are methods described in Cole et al., Monoclonal Antibodies and Cancer Therapy, Alan R. Liss, p. 77 (1985); Boemer et al., J. Immunol., 147(1):86-95 (1991). See also van Dijk and van de Winkel, Curr. Opin. Pharmacol., 2:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE technology). See also, for example, L1 et al., Proc. Natl. Acad. Sci. USA, 103:3557-3562 (2006) regarding human antibodies generated via a human B-cell hybridoma technology.

A “humanized antibody” refers to antibodies made by a non-human cell having variable or variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention can include amino acid residues not encoded by human germline immunoglobulin sequences (e.g., mutations introduced by random or site-specific mutagenesis in vitro or by somatic mutation in vivo), for example in the CDRs. Humanized antibodies can also include antibodies in which CDR sequences derived from the germline of another mammalian species, such as a mouse, have been grafted onto human framework sequences.

Humanized forms of non-human (e.g., murine) antibodies are antibodies that contain minimal sequence derived from non-human immunoglobulin. In some embodiments, a humanized antibody is a human immunoglobulin (recipient antibody) in which residues from a hypervariable region of the recipient are replaced by residues from an hypervariable region of a nonhuman species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies can comprise residues that are not found in the recipient antibody or in the donor antibody. These modifications can be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions can include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally can also include at least a portion of an immunoglobulin constant region (Fc), which can be a human immunoglobulin. Exemplary methods and humanized antibodies include those described by Jones et al. Nature 321:522-525 (1986); Riechmann et al. Nature 332:323-329 (1988); and Presta, Curr. Op. Struct. Biol. 2:593-596 (1992); Vaswani and Hamilton, Ann. Allergy. Asthma & Immunol. 1:105-115 (1998); Harris, Biochem. Soc. Transactions 23:1035-1038 (1995); Burle and Gross, Curr. Op. Biotech. 5:428-433 (1994); and U.S. Pat. Nos. 6,982,321 and 7,087,409.

The term “functional fragment” when used in reference to an antibody refers to a portion of the antibody including heavy or light chain polypeptides that retains some or all of the binding activity as the antibody from which the fragment was derived. Such functional fragments can include, for example, an Fd, Fv, Fab, F(ab′), F(ab)₂, F(ab′)₂, single chain Fv (ScFv), diabody, triabody, tetrabody and minibody. Other functional fragments can include, for example, heavy or light chain polypeptides, variable region polypeptides or CDR polypeptides or portions thereof so long as such functional fragments retain binding activity. Such antibody binding fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Phickthun and Skerra, Meth. Enzymol., 178:497-515 (1989) and in Day, E. D., Advanced Immunochemistry, Second Ed., Wiley-Liss, Inc., New York, NY (1990). Antibody Engineering, Second Edition, Oxford University Press, 1995.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: a, 6 and 7 contain approximately 450 amino acids, while p and F contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (x) of lambda (k) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains can differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. Numbering of amino acid positions used herein is according to the EU Index, as in Kabat et al. (1991)., Sequences of proteins of immunological interest. (U.S. Department of Health and Human Services, Washington, D.C.) 5^{th} Ed. A variable region can be a human variable region.

A CDR refers to one of three hypervariable regions (H1, H2 or H3) within the non-framework region of the immunoglobulin (Ig or antibody) VH j-sheet framework, or one of three hypervariable regions (L1, L2 or L3) within the non-framework region of the antibody VL j-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable), Chothia (structural), or MacCallum (J. Mol. Biol. 262:732-745 (1996)) designations, as set forth in the Table 1 below:

TABLE 1
CDR Definitions

| | Kabat¹ | Chothia ² | MacCallum ³ | Loop Location |
|---|---|---|---|---|
| V_{H} CDR1 | 31-35 | 26-32 | 30-35 | linking B and C strands |
| V_{H} CDR2 | 50-65 | 53-55 | 47-58 | linking C′ and C″ strands |
| V_{H} CDR3 | 95-102 | 96-101 | 93-101 | linking F and G strands |
| V_{L} CDR1 | 24-34 | 26-32 | 30-36 | linking B and C strands |
| V_{L} CDR2 | 50-56 | 50-52 | 46-55 | linking C′ and C″ strands |
| V_{L} CDR3 | 89-97 | 91-96 | 89-96 | linking F and G strands |

¹Residue numbering follows the nomenclature of Kabat et al., supra
² Residue numbering follows the nomenclature of Chothia et al., supra

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “enhance” refers to an increase or improvement in the function or activity of a protein or cell after administration or contacting with a combination described herein compared to the protein or cell prior to such administration or contact.

The term “administering” refers to the act of delivering a combination or composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arteriole, intradermal, subcutaneous, intraperitoneal, intraventricular, and intracranial administration. Administration generally occurs after the onset of the disease, disorder, or condition, or its symptoms but, in some instances, can occur before the onset of the disease, disorder, or condition, or its symptoms (e.g., administration for patients prone to such a disease, disorder, or condition).

The term “coadministration” refers to administration of two or more agents (e.g., a combination described herein and another active agent such as an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compounds described herein can be used in combination with one another, with other active agents known to be useful in treating cancer.

The term “anti-cancer agent” is used in accordance with its plain ordinary meaning and refers to a composition having anti-neoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” refers to a therapy or regimen that includes administration of a chemotherapeutic or anti-cancer agent described herein.

### Combinations

In some embodiments, described herein are combinations (e.g., combination therapies, such as therapeutic methods and uses, kits and compositions) for treating cancer. In some embodiments, the combinations described herein comprise an HDACi and an anti-cancer agent, a PD-1, PD-L1 and/or a CTLA-4 inhibitor. In some embodiments, the HDACi comprises a generic benzamide structure belonging to sub-class I (HDACs 1/2/3 and 8), class II (HDACs 4/5/6/7/9/10) or class IV (HDAC 11) HDACs. In some embodiments, a combination may comprise a first pharmaceutical composition and a second pharmaceutical composition. In some embodiments, the first pharmaceutical composition comprises an HDACi and the second pharmaceutical composition comprises a PD-1 inhibitor. In some embodiments, the first pharmaceutical composition and the second pharmaceutical composition are co-packaged as a kit, which may further include instructions for co-administration of the first and second pharmaceutical compositions. In some embodiments, the first and second compositions may be packaged separately for combination in a clinical setting by administering them to a patient within a time frame during which the patient derives clinical benefit from the first pharmaceutical composition and the second pharmaceutical composition at the same time. In some embodiments, a combination comprises a unit dosage form of a pharmaceutical composition comprising an HDACi and a PD-1 inhibitor. In some embodiments, a combination comprises a first pharmaceutical composition comprising an HDACi for use in the treatment of cancer in combination with a second pharmaceutical composition comprising a PD-1 inhibitor. In some embodiments, a combination comprises a use of an HDACi for preparation of a first pharmaceutical composition for use in the treatment of cancer in combination with a second pharmaceutical composition comprising a PD-1 inhibitor.

### Compositions

Provided herein are combinations (e.g., combination therapies and compositions) useful for treating a variety of diseases, disorders, and symptoms thereof, including for example, cancer. The combinations described herein include an HDAC inhibitor and a PD-L1 inhibitor and/or PD-1 inhibitor, and further a CTLA-4 inhibitor. The combinations described herein also include an HDAC inhibitor and an anti-cancer agent. In one non-limiting example a benzamide HDAC inhibitor of formula I is provided, and examples of PD-L1 inhibitors, PD-1 inhibitors, CTLA-4 inhibitors and anti-cancer agents are described herein. In some embodiments is a combination that includes a therapeutically effective amount of a PD-L1 inhibitor and/or PD-1 inhibitor, a CTLA-4 inhibitor, and a therapeutically effective amount of a compound of formula I:

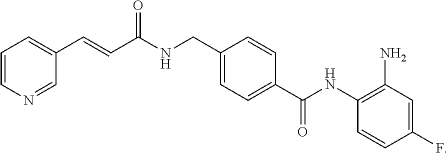 [SMILES: Nc1cc(F)ccc1NC(=O)c1ccc(CNC(=O)/C=C/c2cccnc2)cc1]

Compounds of formula I as described herein include pharmaceutically acceptable salts, pharmaceutically acceptable stereoisomers, prodrugs, enantiomers, diastereomers, hydrates, co-crystals, and polymorphs thereof.

In some instances, the compound of formula I is present at an amount of greater than about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The compound of formula I can be present at an amount greater than about: 1 mg, 2 mg, 3 mg, 4 mg, 5 mg, 6 mg, 7 mg, 8 mg, 9 mg, or 10 mg. In some instances the compound of formula I is present in an amount greater than about 5 mg or about 10 mg. The compound of formula I can be present at an amount greater than about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

The combination can include a compound present in an amount of at least about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of formula I present at an amount of at least about: 1 mg, 2 mg, 3 mg, 4 mg, 5 mg, 6 mg, 7 mg, 8 mg, 9 mg, or 10 mg. In some instances the compound of formula I is present in an amount of at least about 5 mg or about 10 mg. The combination can include a compound of formula I present at an amount of at least about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

The combination can include a compound of formula I present in an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of formula I present at an amount of about: 1 mg, 2 mg, 3 mg, 4 mg, 5 mg, 6 mg, 7 mg, 8 mg, 9 mg, or 10 mg. In some instances the compound of formula I is present in an amount of about 5 mg or about 10 mg. The combination can include a compound of formula I present at an amount of: about 1 mg to about 10 mg, about 1 mg to about 25 mg, about 1 mg to about 50 mg, about 5 mg to about 10 mg, about 5 mg to about 25 mg, about 5 mg to about 50 mg, about 10 mg to about 25 mg, about 10 mg to about 50 mg, about 20 mg to about 50 mg, about 20 mg to about 45 mg, about 20 mg to about 40 mg, about 20 mg to about 35 mg, about 20 mg to about 30 mg, about 50 mg to about 100 mg, or about 100 mg to about 200 mg.

A compound of formula I can be present in the combinations described herein relative to the weight of the patient (e.g., mg/kg). In some instances, the compound of formula I is present in an amount equivalent to about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg. In other instances the compound of formula I is present in an amount equivalent to about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg.

### PD-L1 Inhibitors

PD-L1 inhibitors useful in the combinations described herein include any molecule capable of inhibiting, blocking, abrogating or interfering with the binding of PD-L1 to PD-1, activity or expression of PD-L1. In particular, a PD-L1 inhibitor can be a small molecule compound, a nucleic acid, a polypeptide, an antibody, a peptibody, a diabody, a minibody, a single-chain variable fragment (ScFv), or a functional fragment or variant thereof. In one instance the PD-L1 inhibitor is a small molecule compound (e.g., a compound having a molecule weight of less than about 1000 Da). In some embodiments, the PD-L1 inhibitor is CA-170 (AUPM-170; Curis, Inc.). In other instances, useful PD-L1 inhibitors in the combinations described herein include nucleic acids and polypeptides. The PD-L1 inhibitor can be a polypeptide (e.g., macrocyclic polypeptide) such as those exemplified in U.S. Patent Application Publication No.: 2014/0294898, which is incorporated herein by reference in its entirety and for all purposes. In one example, the PD-L1 inhibitor is an antibody, peptibody, diabody, minibody, ScFv, or a functional fragment thereof. In another example, the PD-L1 inhibitor is a PD-L1 inhibitor antibody. The PD-L1 inhibitor antibody can be a monoclonal or polyclonal antibody. In some embodiments, the PD-L1 inhibitor antibody is a monoclonal antibody.

PD-L1 antibodies include all known types of antibodies and functional fragments thereof, including but not limited to, those exemplified herein such as, for example, human antibodies, mouse antibodies, chimeric antibodies, humanized antibodies, or chimeric humanized antibodies.

In some embodiments, the PD-L1 inhibitor antibody is a human antibody. In another embodiment, the PD-L1 inhibitor antibody is a mouse antibody. In some other embodiments, the PD-L1 inhibitor antibody is a chimeric antibody. In some other embodiments, the PD-L1 inhibitor antibody is a humanized antibody. In some other embodiments, the PD-L1 inhibitor antibody is a chimeric humanized antibody. The PD-L1 inhibitor antibody can be a human antibody or humanized antibody. The PD-L1 inhibitor antibody can be durvalumab, avelumab, atezolizumab, BMS-936559, STI-A1010, STI-A1011, STI-A1012, STI-A1013, STI-A1014, or STI-A1015. In some embodiments, two or more PD-L1 antibodies are administered in combination with a compound of formula I as described herein.

The PD-L1 inhibitor antibody can be durvalumab. Durvalumab is an Fc optimized monoclonal antibody directed against PD-L1, with potential immune checkpoint inhibitory and anti-neoplastic activities. Without being bound by any particular theory, durvalumab binds to PD-L1, thereby blocking its binding to and activation of its receptor, PD-1, which can be expressed on activated T-cells. This can reverse T-cell inactivation and activate the immune system to exert a cytotoxic T-lymphocyte (CTL) response against PD-L1-expressing tumor cells. The Fc region of durvalumab is modified in such a way that it does not induce either antibody-dependent cytotoxicity (ADCC) or complement-dependent cytotoxicity (CDC).

The PD-L1 inhibitor antibody can be avelumab. Avelumab is a human immunoglobulin G1 (IgG1) monoclonal antibody directed against PD-L1, with potential immune checkpoint inhibitory and anti-neoplastic activities. Without being bound by any particular theory, avelumab binds to PD-L1 and prevents the interaction of PD-L1 with its receptor, PD-1. This inhibits the activation of PD-1 and its downstream signaling pathways. This can restore immune function through the activation of cytotoxic T-lymphocytes (CTLs) targeted to PD-L1-overexpressing tumor cells. Avelumab appears to induce an antibody-dependent cellular cytotoxic (ADCC) response against PD-L1-expressing tumor cells.

The PD-L1 inhibitor antibody can be atezolizumab. Atezolizumab is a human, Fc optimized, monoclonal antibody directed against the protein ligand PD-L1, with potential immune checkpoint inhibitory and anti-neoplastic activities. Without being bound by any particular theory, atezolizumab binds to PD-L1, blocking its binding to and activation of its receptor, PD-1, expressed on activated T-cells, which may enhance the T-cell-mediated immune response to neoplasms and reverse T-cell inactivation. In addition, by binding to PD-L1, atezolizumab also appears to prevent binding of PD-L1 to B7.1 expressed on activated T cells, which can further enhance the T-cell-mediated immune response. The Fc region of atezolizumab is modified in such a way that it does not induce either antibody-dependent cytotoxicity (ADCC) or complement-dependent cytotoxicity (CDC).

The PD-L1 inhibitor antibody can be BMS-936559. BMS-936559 is a fully human IgG4 monoclonal antibody directed against PD-L1, with potential immune checkpoint inhibitory activity. Without being bound by any particular theory, BMS-936559 binds to PD-L1 and inhibits its binding to both PD-1 and CD80.

The PD-L1 inhibitor antibody can be STI-A1010, STI-A1011, STI-A1012, STI-A1013, STI-A1014, or STI-A1015. STI-A1010, STI-A1011, STI-A1012, STI-A1013, STI-A1014, and STI-A1015 (Sorrento Therapeutics) are fully human monoclonal antibodies that are each directed against PD-L1. In some other embodiments, the PD-L1 inhibitor can be administered according to established regimens such as those provided in a package insert. PD-1 Inhibitors

PD-1 inhibitors useful in the combinations described herein include any molecule capable of inhibiting, blocking, abrogating or interfering with the activity or expression of PD-1. In particular, a PD-1 inhibitor can be a small molecule compound, a nucleic acid, a polypeptide, an antibody, a peptibody, a diabody, a minibody, a single-chain variable fragment (ScFv), or a functional fragment or variant thereof. In one instance the PD-1 inhibitor is a small molecule compound (e.g., a compound having a molecule weight of less than about 1000 Da.) In other instances, useful PD-1 inhibitors in the combinations described herein include nucleic acids and polypeptides. The PD-1 inhibitor can be a polypeptide (e.g., macrocyclic polypeptide) such as those exemplified in U.S. Patent Application Publication No.: 2014/0294898, which is incorporated herein by reference in its entirety and for all purposes. In one example, the PD-1 inhibitor is an antibody, peptibody, diabody, minibody, ScFv, or a functional fragment thereof. In one example, the PD-1 inhibitor is AMP-224 (GSK).

AMP-224 is a recombinant fusion protein comprising an extracellular domain of the PD-1 ligand programmed cell death ligand 2 (PD-L2) and an Fc region of human IgG. Certain cancers can evade and suppress the immune system, in part, and without being bound by any particular theory by interactions between PD-1 and B7-H1. AMP-224 appears to block this interaction and therefore appears to overcome immune suppression.

In another example, the PD-1 inhibitor is a PD-1 antibody. The PD-1 antibody can be a monoclonal or polyclonal antibody. In some embodiments, the PD-1 antibody is a monoclonal antibody.

PD-1 antibodies include all known types of antibodies and functional fragments thereof, including but not limited to, those exemplified herein such as, for example, human antibodies, mouse antibodies, chimeric antibodies, humanized antibodies, or chimeric humanized antibodies.

In some embodiments, the PD-1 antibody is a human antibody. In some other embodiments, the PD-1 antibody is a mouse antibody. In some other embodiments, the PD-1 antibody is a chimeric antibody. In some other embodiments, the PD-1 antibody is a humanized antibody. In some embodiments, the PD-1 antibody is a chimeric humanized antibody. The PD-1 antibody can be a human antibody or humanized antibody. The PD-1 antibody can be nivolumab, pembrolizumab, pidilizumab, REGN2810, PDR 001, or MEDI0680. In some embodiments, two or more PD-1 antibodies are administered in combination with a compound of formula I as described herein.

The PD-1 antibody can be nivolumab. Nivolumab (marketed as OPDIVO) is a fully human monoclonal antibody directed against PD-1 with immunopotentiation activity. Without being bound by any particular theory, nivolumab binds to and blocks the activation of PD-1 by its cognate ligands, resulting in the activation of T-cells and cell-mediated immune responses against tumor cells or pathogens.

The PD-1 antibody can be pembrolizumab. Pembrolizumab (MK-3475, marketed as KEYTRUDA) is a humanized monoclonal IgG4 antibody directed against human cell surface receptor PD-1 with potential immunopotentiating activity. Without being bound by any particular theory, pembrolizumab binds to PD-1, an inhibitory signaling receptor expressed on the surface of activated T cells, and blocks the binding to and activation of PD-1 by its cognate ligands. The blocking of binding and activity results in the activation of T-cell-mediated immune responses against tumor cells.

The PD-1 antibody can be pidilizumab. Pidilizumab (CT-011) is a humanized monoclonal antibody directed against human PD-1 with immunomodulating and antitumor activities. Without being bound by any particular theory, pidilizumab blocks interaction between the receptor PD-1 with its ligands, resulting in the attenuation of apoptotic processes in lymphocytes, primarily effector/memory T cells, and the augmentation of the anti-tumor activities of NK cells.

The PD-1 antibody can be REGN2810. REGN2810 is a human monoclonal antibody directed against PD-1, with potential immune checkpoint inhibitory and anti-neoplastic activity. Without being bound by any particular theory REGN2810 binds to PD-1, inhibits binding to its cognate ligand, and prevents the activation of its downstream signaling pathways. This can restore immune function through the activation of cytotoxic T-cells.

The PD-1 antibody can be PDR 001. PDR 001 is a fully humanized monoclonal antibody directed against PD-1, with immune checkpoint inhibitory and anti-neoplastic activities. Without being bound by any particular theory, PDR 001 binds to PD-1 expressed on activated T-cells and blocks the interaction with its cognate ligands. The inhibition of ligand binding prevents PD-1-mediated signaling and results in both T-cell activation and the induction of T-cell-mediated immune responses against tumor cells.

The PD-1 antibody can be MEDI0680 (AMP-514) is a monoclonal antibody directed against the PD-1, with potential immunomodulating and anti-neoplastic activity. Without being bound by any particular theory, MEDI0680 appears to inhibit the activation of PD-1 and its downstream signaling pathways. This inhibition can restore immune function through the activation both of T-cells and cell-mediated immune responses against PD-1 overexpressing tumor cells. In some other embodiments, the PD-1 inhibitor can be administered according to established regimens such as those provided in a package insert.

### CTLA-4 Inhibitors

CTLA-4 inhibitors useful in the combinations described herein include any molecule capable of inhibiting, blocking, abrogating or interfering with the activity or expression of CTLA-4. In particular, a CTLA-4 inhibitor can be a small molecule compound, a nucleic acid, a polypeptide, an antibody, a peptibody, a diabody, a minibody, a single-chain variable fragment (ScFv), or a functional fragment or variant thereof. In one instance the CTLA-4 inhibitor is a small molecule compound (e.g., a compound having a molecule weight of less than about 1000 Da.) In other instances, useful CTLA-4 inhibitors in the combinations described herein include nucleic acids and polypeptides. The CTLA-4 inhibitor can be a polypeptide (e.g., macrocyclic polypeptide). In one example, the CTLA-4 inhibitor is an antibody, peptibody, diabody, minibody, ScFv, or a functional fragment thereof. In one example, the CTLA-4 inhibitor is ipilimumab.

In another example, the CTLA-4 inhibitor is a CTLA-4 antibody. The CTLA-4 antibody can be a monoclonal or polyclonal antibody. In some embodiments, the CTLA-4 antibody is a monoclonal antibody.

CTLA-4 antibodies include all known types of antibodies and functional fragments thereof, including but not limited to, those exemplified herein such as, for example, human antibodies, mouse antibodies, chimeric antibodies, humanized antibodies, or chimeric humanized antibodies.

In some embodiments, the CTLA-4 antibody is a human antibody. In other embodiments, the CTLA-4 antibody is a mouse antibody. In some embodiments, the CTLA-4 antibody is a chimeric antibody. In some embodiments, the CTLA-4 antibody is a humanized antibody. In some embodiments, the CTLA-4 antibody is a chimeric humanized antibody. The CTLA-4 antibody can be a human antibody or humanized antibody. The CTLA-4 antibody can be administered in combination with a compound of formula I as described herein. In some other embodiments, the CTLA-4 inhibitor can be administered according to established regimens such as those provided in a package insert.

### CD276 Inhibitors

CD276 (B7-H3) is a relatively newly discovered, but important member of the immune checkpoint family. CD276 is expressed on antigen-presenting cells in active/inflamed “hot” tumor micro-environments (“TMEs”) and suppresses CD8⁺ cytotoxic T cells. CD276 expression is upregulated with administration of a compound of formula I as described herein. CD276 inhibitors useful in the combinations described herein include any molecule capable of inhibiting, blocking, abrogating or interfering with the activity or expression of CD276. In particular, a CD276 inhibitor can be a small molecule compound, a nucleic acid, a polypeptide, an antibody, a peptibody, a diabody, a minibody, a single-chain variable fragment (ScFv), or a functional fragment or variant thereof. In one instance the CD276 inhibitor is a small molecule compound (e.g., a compound having a molecule weight of less than about 1000 Da.) In other instances, useful CD276 inhibitors in the combinations described herein include nucleic acids and polypeptides. The CD276 inhibitor can be a polypeptide (e.g., macrocyclic polypeptide). In one example, the CD276 inhibitor is an antibody, peptibody, diabody, minibody, ScFv, or a functional fragment thereof.

In another example, the CD276 inhibitor is a CD276 antibody. The CD276 antibody can be a monoclonal or polyclonal antibody. In some embodiments, the CD276 antibody is a monoclonal antibody.

CD276 antibodies include all known types of antibodies and functional fragments thereof, including but not limited to, those exemplified herein such as, for example, human antibodies, mouse antibodies, chimeric antibodies, humanized antibodies, or chimeric humanized antibodies.

In some embodiments, the CD276 antibody is a human antibody. In some embodiments, the CD276 antibody is a mouse antibody. In some embodiments, the CD276 antibody is a chimeric antibody. In some embodiments, the CD276 antibody is a humanized antibody. In some embodiments, the CD276 antibody is a chimeric humanized antibody. The CD276 antibody can be a human antibody or humanized antibody. The CD276 antibody can be administered in combination with a compound of formula I as described herein, or with any of the other compositions described herein. In some other embodiments, the CD276 antibody can be administered according to established regimens such as those provided in a package insert.

A PD-L1 inhibitor antibody, PD-1 inhibitor antibody, CTLA-4 inhibitor antibody, and/or CD276 inhibitor antibody (any one of which is referred to as “Inhibitor Antibody” herein) can be of any antibody isotype. The term isotype refers to the antibody class that is encoded by heavy chain constant region genes. The heavy chains of a given antibody or functional fragment determine the class of that antibody or functional fragment: IgM, IgG, IgA, IgD or IgE. Each class can have either κ or λ. light chains. The term subclass refers to the minor differences in amino acid sequences of the heavy chains that differentiate the subclasses. In humans there are two subclasses of IgA (subclasses IgA1 and IgA2) and there are four subclasses of IgG (subclasses IgG1, IgG2, IgG3 and IgG4). Such classes and subclasses are well known to those skilled in art.

Useful Inhibitor Antibodies bind to their substrates with sufficient strength to inhibit activity of the substrate (e.g., PD-L1, PD-1, CTLA-4, and/or CD276). The term “bind” as used herein refers to an interaction between molecules to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions or forces. Binding of an antibody or functional fragment thereof can be detected using, for example, an enzyme-linked immunosorbent assay or any one of a number of methods that are well known to those skilled in the art.

The strength of the total non-covalent interactions between a single antigen-binding site on an Inhibitor Antibody or functional fragment and a single epitope of a target molecule is the affinity of the antibody or functional fragment for that epitope. The ratio of association (k_{I}) to dissociation (k_{-I}) of an antibody or functional fragment thereof to a monovalent antigen (k_{I}/k_{-I}) is the association constant K, which is a measure of affinity. The value of K varies for different complexes of antibody or functional fragment and antigen and depends on both k_{I} and k_{-I}. The association constant K for an antibody or functional fragment of the invention can be determined using any method provided herein or any other method well known to those skilled in the art.

The affinity at one binding site does not always reflect the true strength of the interaction between an antibody or functional fragment and an antigen. When complex antigens containing multiple, repeating antigenic determinants come in contact with antibodies containing multiple binding sites, the interaction of such an antibody or functional fragment with antigen at one site will increase the probability of a reaction at a second site. The strength of such multiple interactions between a multivalent antibody and antigen is called the avidity. The avidity of an antibody or functional fragment can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The specificity of an Inhibitor Antibody or functional fragment thereof refers to the ability of an individual antibody or functional fragment thereof to react with only one antigen (e.g., a single epitope of PD-L1, PD-1, and CTLA-4). An antibody or functional fragment can be considered specific when it can distinguish differences in the primary, secondary or tertiary structure of an antigen or isomeric forms of an antigen.

The Inhibitor Antibody can be present in an amount as a measure with regards to the weight of the patient in need thereof. For example, the Inhibitor Antibody can be present in an amount of about: 0.1 mg/kg to about 50 mg/kg, 0.1 mg/kg to about 40 mg/kg, 0.1 mg/kg to about 30 mg/kg, 0.1 mg/kg to about 25 mg/kg, 0.1 mg/kg to about 20 mg/kg, 0.1 mg/kg to about 15 mg/kg, 0.1 mg/kg to about 10 mg/kg, 0.1 mg/kg to about 7.5 mg/kg, 0.1 mg/kg to about 5 mg/kg, 0.1 mg/kg to about 2.5 mg/kg, or about 0.1 mg/kg to about 1 mg/kg. The Inhibitor Antibody can be present in an amount of about: 0.5 mg/kg to about 50 mg/kg, 0.5 mg/kg to about 40 mg/kg, 0.5 mg/kg to about 30 mg/kg, 0.5 mg/kg to about 25 mg/kg, 0.5 mg/kg to about 20 mg/kg, 0.5 mg/kg to about 15 mg/kg, 0.5 mg/kg to about 10 mg/kg, 0.5 mg/kg to about 7.5 mg/kg, 0.5 mg/kg to about 5 mg/kg, 0.5 mg/kg to about 2.5 mg/kg, or about 0.5 mg/kg to about 1 mg/kg. The Inhibitor Antibody can be present in an amount of about 0.5 mg/kg to about 5 mg/kg or about 0.1 mg/kg to about 10 mg/kg. The Inhibitor Antibody can be present in an amount of about 0.1 mg/kg to about 20 mg/kg or about 0.1 mg/kg to about 30 mg/kg.

In still some other embodiments, the Inhibitor Antibody can be present at an amount of about: 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, or 50 mg/kg. The Inhibitor Antibody can be present at an amount of about: 1 mg/kg, 2 mg/kg, 3 mg/kg, 5 mg/kg, 10 mg/kg, 15 mg/kg, 20 mg/kg, 25 mg/kg, or 30 mg/kg. The Inhibitor Antibody can be present at an amount of about: 3 mg/kg, 10 mg/kg, 20 mg/kg, or 30 mg/kg.

The Inhibitor Antibody can be present in the combination at an amount of about: 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, or 200 mg. The Inhibitor Antibody can be present in the combination at an amount of about: 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. The Inhibitor Antibody can be present in the combination at an amount of about 1000 mg to about 2000 mg. The Inhibitor Antibody can be present in the combination at an amount of about: 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 to about 2000 mg.

The Inhibitor Antibody can be present in the combination in an amount of about 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL, or 500 mg/mL. In some embodiments, the Inhibitor Antibody is present in the combination in an amount of about: 1 mg/mL to about 10 mg/mL, 5 mg/mL to about 10 mg/mL, 5 mg/mL to about 15 mg/mL, 10 mg/mL to about 25 mg/mL; 20 mg/mL to about 30 mg/mL; 25 mg/mL to about 50 mg/mL, or 50 mg/mL to about 100 mg/mL.

In some instances the therapeutically effective amount of an Inhibitor Antibody is determined as an amount provided in a package insert provided with the Inhibitor Antibody. The term package insert refers to instructions customarily included in commercial packages of medicaments approved by the FDA or a similar regulatory agency of a country other than the USA, which contains information about, for example, the usage, dosage, administration, contraindications, and/or warnings concerning the use of such medicaments.

Compounds of formula I as described herein can be provided in amounts that are synergistic with the amount of the PD-L1 and/or PD-1 inhibitor, and a CTLA-4 inhibitor. The term synergistic refers to a combination described herein (e.g., a compound of formula I and a PD-L1 and/or PD-1 inhibitor, plus a CTLA-4 inhibitor—including co-administration with another active agent such as an anti-cancer agent described herein) or a combination of regimens such as those described herein that is more effective than the additive effects of each individual therapy or regimen.

A synergistic effect of a combination described herein can permit the use of lower dosages of one or more of the components of the combination (e.g., a compound of formula I, or a PD-L1 inhibitor, or a PD-1 inhibitor, or a CTLA-4 inhibitor). A synergistic effect can permit less frequent administration of at least one of the administered therapies (e.g., a compound of formula I, or a PD-L1 inhibitor, or a PD-1 inhibitor, or a CTLA-4 inhibitor or an anti-cancer agent) to a subject with a disease, disorder, or condition described herein. Such lower dosages and reduced frequency of administration can reduce the toxicity associated with the administration of at least one of the therapies (e.g., a compound of formula I, or a PD-L1 inhibitor, or a PD-1 inhibitor, or a CTLA-4 inhibitor or an anti-cancer agent) to a subject without reducing the efficacy of the treatment. A synergistic effect as described herein avoid or reduce adverse or unwanted side effects associated with the use of any therapy.

### Pharmaceutical Compositions

Combinations described herein can be provided as a pharmaceutical composition suitable for administration via any route to a patient described herein including but not limited to: oral, mucosal (e.g., nasal, inhalation, pulmonary, sublingual, vaginal, buccal, or rectal), parenteral (e.g., subcutaneous, intravenous, bolus injection, intramuscular, or intra-arterial), topical (e.g., eye drops or other ophthalmic preparations), transdermal or transcutaneous administration to a patient.

Exemplary of dosage forms include: tablets; caplets; capsules (e.g., gelatin capsules); cachets; lozenges; suppositories; powders; gels; liquid dosage forms suitable for parenteral administration to a patient; and sterile solids (e.g., crystalline or amorphous solids) that can be reconstituted to provide liquid dosage forms suitable for parenteral administration to a patient.

Pharmaceutical compositions and dosage forms described herein typically include one or more excipients. Suitable excipients are well known to those skilled in the art of pharmacy. Whether a particular excipient is suitable for incorporation into a pharmaceutical composition or dosage form depends on a variety of factors such as, for example, the intended route of administration to the patient. Pharmaceutical compositions described herein can include other agents such as stabilizers, lubricants, buffers, and disintegrants that can reduce the rate by which an active ingredient can decompose in a particular formulation.

Pharmaceutical compositions described herein can in some instances include additional active agents other than those in the combinations described herein (e.g., an anti-cancer agent such as those described herein) in an amount provided herein.

In some embodiments, the compound of formula I is provided in an oral dosage form such as a tablet or capsule. In some embodiments, the compound of formula I is supplied as a powder (e.g., lyophilized powder) that can be resuspended in a liquid suitable for parenteral administration.

PD-L1 inhibitors, PD-1 inhibitors, and CTLA-4 inhibitors described herein can be provided in forms convenient to or facilitate their administration to a patient. For example, where the inhibitor is an Inhibitor Antibody as described herein, the inhibitor can be formulated as a ready to use solution for parenteral administration. In other examples, the inhibitor, including for example an Inhibitor Antibody, can be formulated as a powder (e.g., lyophilized powder) that can be resuspended in a liquid suitable for parenteral administration. In some embodiments, the combination includes an Inhibitor Antibody formulated for intravenous administration. In some other embodiments the combination includes a compound of formula I formulated as an oral dosage form (e.g., a tablet or capsule) and an Inhibitor Antibody formulated for intravenous administration.

Combinations described herein can be provided as controlled release pharmaceutical products, which have a goal of improving drug therapy over that achieved by their non-controlled counterparts. Controlled release formulations can extend activity of the drug, reduce dosage frequency, and increase subject compliance. In addition, controlled release formulations can be used to affect the time of onset of action or other characteristics, such as blood levels of the drug, and can thus affect the occurrence of side (e.g., adverse) effects.

### Kits

The combinations and pharmaceutical compositions described herein can be provided as part of a kit. Such kits can, for example, improve patient compliance or improve the accuracy or ease of preparation for administering the combination. The kit includes a compound of formula I where the compound is supplied in a formulation as described herein.

Kits of the invention can include the combinations described herein having the same or different formulation. Each component of a combination described herein in a kit can be supplied in a separate, individual container. Alternatively or additionally, components of the combinations described herein can be supplied in a single container. In such instances, the container can be a container that is ready for administration to a patient in need thereof, such as for example, an IV bag, ampoule, or a syringe. In some embodiments, the compound of formula I in the kit is formulated for oral administration (e.g., a tablet, capsule, or sachet).

The contents of kits described herein can be provided in sterile form. The kit and its contents can be provided in a form that is ready for administration to the subject in need. In such instances, the components of the combination of the kit are supplied as a formulation and optionally in an administration device such that administration requires little to no further action by the user. Where kits include administration devices, such devices include devices known and understood by those skilled in the art for routes of administration described herein, such as but not limited to, syringes, pumps, bags, cups, inhalers, droppers, patches, creams, or injectors.

### Method

The combinations, pharmaceutical compositions, and kits described herein are useful for treating diseases, disorders, or alleviating or eliminating the symptoms of diseases and disorders such as, for example, cancer. It is to be understood that the methods described herein pertain to administration of combinations and pharmaceutical compositions described herein, and such combinations and pharmaceutical compositions can be provided in the form of a kit as described herein. Provided herein are methods of treating cancer by administering a therapeutically effective amount of a combination described herein to a patient in need thereof. Also provided herein are methods of managing cancer by administering therapeutically effective amount of a combination described herein to a patient in need thereof.

In some embodiments, the combination is used to treat cancer. In some embodiments, the cancer is a cancer described herein.

In some embodiments, the combination is an HDAC inhibitor (HDACi), a PD-L1 inhibitor, and a CTLA-4 inhibitor. In some embodiments, the combination is an HDAC inhibitor (HDACi), a PD-1 inhibitor, and a CTLA-4 inhibitor. In some other embodiments, the combination is an HDAC inhibitor (HDACi), a PD-1 inhibitor and an anti-cancer agent.

Combinations useful in the methods described herein include a compound of formula I:

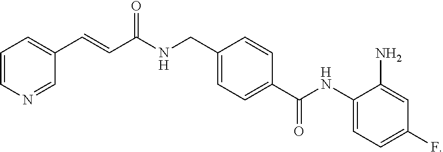 [SMILES: Nc1cc(F)ccc1NC(=O)c1ccc(CNC(=O)/C=C/c2cccnc2)cc1]

The PD-L1 inhibitors, PD-1 inhibitors, and CTLA-4 inhibitors for use in the methods described herein are those inhibitors described herein. For example, the PD-L1 inhibitors, PD-1 inhibitors, and CTLA-4 inhibitors can be a small molecule compound, a nucleic acid, a polypeptide, an antibody, a peptibody, a diabody, a minibody, a single-chain variable fragment (ScFv), or functional fragment or variant thereof. In other examples, the inhibitor can be an Inhibitor Antibody as set forth above.

### Target Cancers

The cancer can be a solid tumor. The cancer can be a hematological cancer. In some instances, the cancer is a solid tumor such as squamous cell carcinoma, non-squamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST).

In some embodiments, the cancer is a solid tumor such as non-small cell lung cancer (NSCLC), hepatocellular carcinoma, melanoma, ovarian cancer, breast cancer, pancreatic cancer, renal cell carcinoma, or colorectal cancer. The cancer can be non-small cell lung cancer (NSCLC). The cancer can be hepatocellular carcinoma. The cancer can be melanoma. The cancer can be ovarian cancer. The cancer can be breast cancer. The cancer can be pancreatic cancer. The cancer can be renal cell carcinoma. The cancer can be colorectal cancer.

Provided herein are methods of treating NSCLC by administering a therapeutically effective amount of a combination described herein where the combination includes a compound of formula I and an Inhibitor Antibody. In some embodiments, the NSCLC is Stage IIA or Stage IIB. The NSCLC can be a Stage IIIA or Stage IIIB cancer. The NSCLC can be a Stage IV cancer. Staging of cancers as described herein is described by the American Joint Committee on Cancer TNM classification of malignant tumors cancer staging notation as is well understood in the art. Those of skill in the art will readily understand other staging classification systems are available and applicable to the methods described herein. In some instances, the method is a method of treating Stage IIIA or IIIB NSCLC by administering a combination described herein that includes a compound of formula I and an Inhibitor Antibody.

Still further provided herein are methods of treating melanoma by administering a therapeutically effective amount of a combination described herein where the combination includes a compound of formula I and an Inhibitor Antibody. In some embodiments the melanoma is a Stage IIA, IIB, or IIC cancer. In some embodiments, the melanoma is a Stage IIIA, Stage IIIB, or Stage IIIC cancer. In some other embodiments, the melanoma is a Stage IV cancer. In some embodiments the method is a method of treating Stage II (e.g., Stage IIA, IIB, or IIC) melanoma by administering a therapeutically effective amount of a combination described herein where the combination includes a compound of formula I and an Inhibitor Antibody.

Also provided herein are methods of treating breast cancer by administering a therapeutically effective amount of a combination described herein where the combination includes a compound of formula I and an Inhibitor Antibody. The breast cancer can be HER2 negative breast cancer. The breast cancer can be a HER2 positive breast cancer. The breast cancer can be triple-negative breast cancer. In some embodiments the breast cancer is a Stage IA or Stage D3 cancer. In some embodiments, the breast cancer is a Stage IIA or Stage IIB cancer. In some embodiments, the breast cancer is a Stage IIIA, Stage IIIB, or Stage IIIC cancer. In some embodiments, the breast cancer is a Stage IV cancer.

In other embodiments, the cancer is a hematological cancer such as lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CML), acute lymphocytic leukemia, (ALL), or chronic lymphocytic leukemia (CLL). In some embodiments, the cancer is Hodgkin's Lymphoma or Reed-Sternberg disease.

The combinations described herein can be administered to a cancer patient at any time following diagnosis. For example, the cancer patient can be treatment naive (e.g., has not received a cancer therapy for the diagnosed cancer). The cancer patient can be treatment naive for one cancer but can be diagnosed with one or more other cancers resulting from, for example, metastasis or malignancy. The cancer patient can be immune checkpoint naive for one or more cancers. The cancer patient can have a cancer that is refractory. In some instances, the combinations described herein are administered as a first line therapy (e.g., the first therapy administered to a treatment naive cancer patient) to a patient in need thereof.

However, cancer morbidity and mortality is often associated with ineffective therapy or a cancer gaining resistant to or becoming refractory to one or more cancer therapies. The combinations described herein can, therefore, be administered to patients in need thereof as a second, third, fourth, fifth, sixth, or more line of treatment. The combinations described herein can be administered to a cancer patient who has been treated with at least one anti-cancer therapy or anti-cancer agent. In some instances the patient has received at least one anti-cancer therapy including, for example, chemotherapy, radiotherapy, surgery, targeted therapy, immunotherapy, or a combination thereof. The patient can have a cancer that is resistant/refractory to treatment with at least one anti-cancer agent.

The methods of treating cancers herein include treating subjects who have been treated with a checkpoint inhibitor and have experienced no response to treatment, or a partial response, or stable disease, but then develop resistance to treatment with progression of disease or who have experienced a complete response to treatment, but then develop resistance to treatment with progression of disease (as defined by RECIST or other criteria). Resistance is defined as disease progression during treatment or a lack of response to treatment. Such Inhibitor Antibody treatment failures can be treated with an Inhibitor Antibody in combination with an HDAC inhibitor, such as, without limitation, HBI-8000 or an HDAC inhibitor that inhibits cancer-associated Class I HDAC selected from one or more of HDAC1, HDAC2, or HDAC3. In some instances the HDAC inhibitor also inhibits Class IIb HDAC1.

### Response Criteria

### RECIST:

RECIST is a set of established criteria or standards, internationally recognized for evaluating patient response, stability and progression in clinical trials and in the clinical practice. Originally published in 2000, and revised in 2009 (Eisenhauer E A, et al.; New response criteria in solid tumors: revised RECIST guideline (version 1.1); Eur. J. Cancer 2009; 45:228-47), as a joint effort of the European Organization for Research and Treatment of Cancer, the National Cancer Institute of the United States and the National Cancer Institute of Canada Clinical Trials Group, RECIST has traditionally been utilized in the evaluation of response to chemotherapy.

### Evaluation of Target Lesions:

Complete Response (CR): Disappearance of all target lesions; Partial Response (PR): At least a 30% decrease in the sum of the LD (longest diameter) of target lesions, taking as reference the baseline sum LD; Stable Disease (SD): Neither sufficient shrinkage to qualify for PR nor sufficient increase to qualify for PD, taking as reference the smallest sum LD since the treatment started; Progressive Disease (PD): At least a 20% increase in the sum of the LD of target lesions, taking as reference the smallest sum LD recorded since the treatment started or the appearance of one or more new lesions.

### Evaluation of Non-Target Lesions:

Complete Response (CR): Disappearance of all non-target lesions and normalization of tumor marker level; Incomplete Response/Stable Disease (SD): Persistence of one or more non-target lesion(s) or/and maintenance of tumor marker level above the normal limits; Progressive Disease (PD): Appearance of one or more new lesions and/or unequivocal progression of existing non-target lesions.

### Other Response Criteria:

Other response criteria include the Immune-Related Response Criteria or iRECIST, as defined by Wolchok et al., in 2009 (Wolchok J D, et al.; Guidelines for the Evaluation of Immune Therapy Activity in Solid Tumors: Immune-Related Response Criteria. Clin. Cancer Res 2009; 15(23):7412-20) and the revised International Working Group Response Criteria (Cheson B D et al., Revised response criteria for malignant lymphoma. J. Clin. Oncol. 2007; 25:579-586).

### Evaluation of the Effect of HDACi on Heart Rate:

In conjunction with the pharmacokinetic analysis, Holter monitors were used to record 12-lead continuous digital ECGs (under controlled conditions to minimize digital noise) on a baseline day (within 1-7 days prior to C1D1) and on C1D1 (Phase 1b only). Triplicate 10-second ECGs were extracted from the Holter flashcard at matched time-points on the baseline day and on C1D1 at pre-dose (0.5 hr prior to breakfast), and up to 4 hours post-dose. The PR, QRS, RR, and QT intervals were analyzed by a central ECG laboratory. Mealtimes (breakfast) were standardized across both days. The measured QT data were corrected for heart rate using the Fridericia correction (QTcF).
QTcF was calculated using the formula:
   Fridericia: QTcFri=QT/(RR)^{1/3} (Fridericia L S. Die systolendauer im elektrokardiogramm bei normalenmenschen und bei herzkranken. Acta Med Scand. 1920; 53:469-486).

The methods of treating cancers herein include treating subjects with an HDACi such that administration of the HDACi causes no increase in QTc, QTcF, or heart rate (HR).

The methods of treating cancer include methods for inhibiting cell growth by administering a therapeutically effective amount of a combination described herein where the combination includes a compound of formula I and a PD-L1 inhibitor and/or PD-1 inhibitor, plus a CTLA-4 inhibitor described herein.

Also provided herein are methods of inhibiting metastasis of a cancer in a patient in need thereby by administering a therapeutically effective amount of a combination described herein where the combination includes a compound of formula I and a PD-L1 inhibitor and/or PD-1 inhibitor, plus a CTLA-4 inhibitor described herein. In some embodiments, metastasis is inhibited by at least about 5%, 10%, 20%, 30%, 40%, 50%, 60%, 70%, 80%, 90%, or 100%.

In some embodiments is a method of reducing pre-existing tumor metastasis in a cancer patient in need thereof by administering a therapeutically effective amount of a combination described herein where the combination includes a compound of formula I and a PD-L1 inhibitor and/or PD-1 inhibitor, plus a CTLA-4 inhibitor described herein. In some embodiments, pre-existing tumor metastasis is reduced by at least about 5%, 10%, 20%, 30%, 40%, 50%, 60%, 70%, 80%, 90%, or 100%.

In some other embodiments the methods of treating cancer also provide for methods for reducing tumor burden in an individual by administering a therapeutically effective amount of a combination described herein where the combination includes a compound of formula I and a PD-L1 inhibitor and/or PD-1 inhibitor, plus a CTLA-4 inhibitor described herein. In some embodiments, tumor burden is reduced by at least about 5%, 10%, 20%, 30%, 40%, 50%, 60%, 70%, 80%, 90%, or 100%.

In some embodiments the methods of treating cancer also provide for methods for reducing tumor burden in an individual by administering a therapeutically effective amount of a combination described herein where the combination includes a compound of formula I and a PD-L1 inhibitor and/or PD-1 inhibitor, plus a CTLA-4 inhibitor described herein. In some embodiments, tumor burden is reduced by at least about 5%, 10%, 20%, 30%, 40%, 50%, 60%, 70%, 80%, 90%, or 100%.

The methods of treating cancer described herein also provide for methods for increasing or otherwise prolonging time to disease progression of certain stages (including advanced stages of cancer such as Stage III and IV cancer described herein). Time to disease progression can be prolonged in a patient by administering a therapeutically effective amount of a combination described herein where the combination includes a compound of formula I and a PD-L1 inhibitor and/or PD-1 inhibitor, plus a CTLA-4 inhibitor described herein. In some embodiments, the increase is a comparison between the time to disease progression without treatment and with treatment with a combination described herein. In some embodiments, the methods described herein prolong the time to disease progression by at least 1 week, 2 weeks, 3 weeks, 4 weeks, 1 month, 2 months, 3 months, 4 months, 5 months, 6 months, 7 months, 8 months, 9 months, 10 months, 11 months, 1 year, or more, including values therein.

The methods of treating cancer described herein also provide for methods for increasing or otherwise prolonging survival (including overall survival) of patients diagnosed with cancer as described herein. Patient survival can be prolonged by administering a therapeutically effective amount of a combination described herein where the combination includes a compound of formula I and a PD-L1 inhibitor and/or PD-1 inhibitor, plus a CTLA-4 inhibitor described herein. In some embodiments, the increase is a comparison between the survival without treatment and with treatment with a combination as described herein. In some embodiments, the methods described herein prolong survival by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

The methods of treating cancer described herein also provide for methods for increasing progression-free survival of patients diagnosed with cancer as described herein. Patient progression-free survival can be prolonged by administering a therapeutically effective amount of a combination described herein where the combination includes a compound of formula I and a PD-L1 inhibitor and/or PD-1 inhibitor, plus a CTLA-4 inhibitor described herein. In some embodiments, the increase is a comparison between the progression-free survival without treatment and with treatment with a combination as described herein. In some embodiments, the methods described herein increase progression-free survival by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

Also provided herein are methods of reducing a level of myeloid-derived suppressor cells (MDSC) in a patient in need thereof by administering an effective amount of a combination described herein where the combination includes a compound of formula I and a PD-L1 inhibitor and/or PD-1 inhibitor, plus a CTLA-4 inhibitor described herein. The reduction of MDSC can benefit the treatment of a cancer described herein. The level of MDSC in a human patient can be measured before, during, and after administration of a combination described herein. In some embodiments, it can be useful to compare pre- and post-administration amounts of MDSC in the patient. A reduction in the amount, level, or number of MDSC following administration can indicate effectiveness of the combination in, for example, treating a cancer described herein. MD SC levels can be monitored over the course of a treatment or regimen described herein with a combination described herein. In such instances, the determination of MD SC levels at various points during the course of administration can indicate the effectiveness of the regimen.

Methods of reducing the percentage or level of Treg cells in a patient in need thereof are also provided herein. Such methods include administering an effective amount of a combination described herein where the combination includes a compound of formula I and a PD-L1 inhibitor and/or PD-1 inhibitor, plus a CTLA-4 inhibitor described herein. The reduction of Treg cells can benefit the treatment of a cancer described herein. The level of Treg cells in a human patient can be measured before, during, and after administration of a combination described herein. In some embodiments, it can be useful to compare pre- and post-administration amounts of Treg cells in the patient. A reduction in the amount, level, or number of Treg cells following administration can indicate effectiveness of the combination in, for example, treating a cancer described herein. Treg cell levels can be monitored over the course of a treatment or regimen described herein with a combination described herein. In such instances, the determination of Treg cell levels at various points during the course of administration can indicate the effectiveness of the regimen.

The combinations described herein can be useful in methods of enhancing activity of natural killer (NK) cells. The combinations described herein can also be useful in methods of enhancing activity of cytotoxic T-cells. The methods of enhancing include contacting a NK cell or cytotoxic T-cell with a combination described herein where the combination enhances the activity of the NK cell or cytotoxic T-cell relative to its activity prior to the contact. In some embodiments, the enhanced activity of the NK cell or cytotoxic T-cell is in a cancer patient who has been administered a combination as described herein.

The combinations described herein can also enhance antibody-dependent cell-mediated cytotoxicity in a cancer patient upon administration of a combination as described herein.

The combinations described herein can include administration of each therapy (e.g., a compound of formula I and a PD-L1 inhibitor and/or PD-1 inhibitor, plus a CTLA-4 inhibitor), where the administration is performed simultaneously or sequentially (in either order). In some embodiments, the compound of formula I and the PD-L1 inhibitor and/or PD-1 inhibitor, plus a CTLA-4 inhibitor are administered simultaneously (e.g., within at least 1 to 5 min of each other). In some embodiments, the compound of formula I and the PD-L1 inhibitor and/or PD-1 inhibitor, plus a CTLA-4 inhibitor are administered sequentially (e.g., within at least 10 min, 15 min, 30 min, 1 h, 2 h, 5 h, 10 h, 12 h, 1 day, 2 days, 5 days, 7 days, 14 days, or 21 days of each other).

The compound of formula I can be administered, for example, once a day (QD), twice daily (BID), once a week (QW), twice weekly (BIW), three times a week (TIW), or monthly (QM) regularly on a continuous base or intermittent base such as BIW for 3 months then resume a month later. For example, the compound of formula I can be administered BID. The compound of formula I can be administered TIW. In some instances, the compound of formula I is administered 2 to 3 times a week. In some embodiments, the compound of formula I is administered QD. The compound can be administered QD for about: 1 day to about 7 days, 1 day to about 14 days, 1 day to about 21 days, 1 day to about 28 days, or daily until disease progression or unacceptable toxicity. The administration of a compound of formula I can, in part, depend upon the tolerance of the patient where greater tolerance can allow greater or more frequent administration. Alternatively, where a patient shows poor tolerance to a compound of formula I, a less amount of the compound or a less frequent dosing can be performed. Compounds of formula I can be administered in any regimen as described herein.

For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QD. For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, BIW. For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, TIW. For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QW. For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, Q2W. For example, a compound of formula I can be administered at an amount of about 5 mg or about 10 mg, QD. For example, a compound of formula I can be administered at an amount of about 5 mg or about 10 mg, BIW. For example, a compound of formula I can be administered at an amount of about 5 mg or about 10 mg, TIW. For example, a compound of formula I can be administered at an amount of about 5 mg or about 10 mg, QW. For example, a compound of formula I can be administered at an amount of about 5 mg or about 10 mg, Q2W. Administration of a compound of formula I can be continuous. Administration of a compound of formula I can be intermittent.

For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QD. For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, BIW. For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, TIW. For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QW. For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, Q2W. Administration of a compound of formula I can be continuous.

### Administration of a Compound of Formula I can be Intermittent.

For example, a compound of formula I can be administered at an amount of about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QD. For example, a compound of formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, BIW. For example, a compound of formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, TIW. For example, a compound of formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QW. For example, a compound of formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, Q2W. In one example, a compound of formula I can be administered at an amount of about 15 mg/kg to about 75 mg/kg, QD. In another example, a compound of formula I can be administered at an amount of about 20 mg/kg to about 50 mg/kg. In still another example, a compound of formula I can be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a compound of formula I can be continuous.

### Administration of a Compound of Formula I can be Intermittent.

For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QD. For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, BIW. For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, TIW. For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QW. For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, Q2W. In one example, a compound of formula I can be administered at an amount of about 15 mg/kg to about 75 mg/kg, QD. In another example, a compound of formula I can be administered at an amount of about 20 mg/kg to about 50 mg/kg. In still another example, a compound of formula I can be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a compound of formula I can be continuous. Administration of a compound of formula I can be intermittent.

As used herein, the term daily is intended to mean that a therapeutic compound of a combination described herein, such as a compound of formula I, is administered once or more than once each day for a period of time. The term continuous is intended to mean that a therapeutic compound of a combination described herein, such as a compound of formula I, is administered daily for an uninterrupted period of at least 10 days to 52 weeks. The term intermittent or intermittently as used herein is intended to mean stopping and starting at either regular or irregular intervals. For example, intermittent administration of a therapeutic compound of a combination described herein, such as a compound of formula I, includes administration for one to six days per week (e.g., 2 to 3 times per week or QD), administration in cycles (e.g., daily administration for two to eight consecutive weeks, then a rest period with no administration at least one day), or, for example, administration on alternate days.

Where the inhibitor is an Inhibitor Antibody, it can be administered according to established regimens such as those provided in a package insert. The Inhibitor Antibody can be administered in an amount described herein and can be administered QW, once every 2 weeks (Q2W), once every 3 weeks (Q3W), or once every 4 weeks (Q4W). In some embodiments, the Inhibitor Antibody is administered Q2W or Q4W. In some other embodiments, the Inhibitor Antibody is administered Q2W. In some embodiments, the Inhibitor Antibody is administered Q3W. In some embodiments, the Inhibitor Antibody is administered BIW for at least 3 weeks. In some embodiments, the Inhibitor Antibody is administered Q4W.

For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), QW. For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), Q2W. For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), Q4W. For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), B4W (twice every 4 weeks). For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), Q3W. For example, the Inhibitor Antibody can be administered at an amount of about 1000 mg to about 2000 mg (including for example 1000 mg, 1100 mg, 1200 mg, 1300 mg, 1400 mg, 1500 mg, 1600 mg), Q2W. For example, the Inhibitor Antibody can be administered at an amount of about 1000 mg to about 2000 mg (including for example 1000 mg, 1100 mg, 1200 mg, 1300 mg, 1400 mg, 1500 mg, 1600 mg), Q3W. For example, the Inhibitor Antibody can be administered at an amount of about 1000 mg to about 2000 mg (including for example 1000 mg, 1100 mg, 1200 mg, 1300 mg, 1400 mg, 1500 mg, 1600 mg), Q4W. Administration of the Inhibitor Antibody can be continuous. Administration of the Inhibitor Antibody can be intermittent.

The Inhibitor Antibody can be administered as an intravenous infusion over about 10, 20, 30, 40, 50, or 60 or more minutes. The Inhibitor Antibody can be administered as an intravenous infusion over about 60 minutes once every 1, 2, 3, 4, 5 or more weeks. The Inhibitor Antibody can be administered as an intravenous infusion over about 60 minutes once every two weeks. The Inhibitor Antibody can be administered as an intravenous infusion over about 60 minutes once every three weeks. The Inhibitor Antibody can be administered as an intravenous infusion over about 60 minutes once every four weeks. The Inhibitor Antibody can be administered as an intravenous infusion according to a package insert. Administration of Inhibitor Antibody can be continuous.

### Administration of Inhibitor Antibody can be Intermittent.

The combinations described herein can be administered in a regimen. The regimen can be structured to provide therapeutically effective amounts of a compound of formula I and an inhibitor, such as an Inhibitor Antibody, over a predetermined period of time (e.g., an administration time). The regimen can be structured to limit or prevent side-effects or undesired complications of each of the components of the combination described herein. The regimen can be structured in a manner that results in increased effect for both therapies of the combination (e.g., synergy). Regimens useful for treating cancer can include any number of days of administration which can be repeated as necessary. Administration periods can be broken by a rest period that includes no administration of at least one therapy. For example, a regimen can include administration periods that include 2, 3, 5, 7, 10, 15, 21, 28, or more days. These periods can be repeated. For example, a regimen can include a set number of days as previously described where the regimen is repeated 1, 2, 3, 4, 5, 6, 7, 8, 9, 10, 11, 12, 13, or more times.

Regimens can include a rest period of at least 1, 2, 3, 5, 7, 10, or more days, where at least one therapy is no longer administered to a patient. The rest period can be determined by, for example, monitoring the reaction of the patient to the drug or by measuring the efficacy of the treatment. A rest period can be applicable to a single therapy, such that only one therapy of a combination described herein is discontinued in the rest period but the other therapy(ies) are still administered. Rest periods can be applied to all of the therapies administered to the subject such that the subject receives no therapy for a set period of time during the rest period.

Regimens described herein for the treatment of cancer using the combinations described herein can be continued until disease progression or unacceptable toxicity.

Regimens for administration of combinations described herein include, for example administration of a compound of formula I BIW or TIW and administration of a PD-L1 and/or PD-1 inhibitor, plus CTLA-4 inhibitor. For example, a compound of formula I can be administered QD for about 21 days and an Inhibitor Antibody described herein can be administered Q2W or Q4W). For example, a compound of formula I can be administered BIW or TIW and an Inhibitor Antibody described herein can be administered Q2W. In another exemplary regimen, a compound of formula I can be administered BIW or TIW and an Inhibitor Antibody can be administered BIW for 2 or 3 weeks. In still another exemplary regimen, a compound of formula I can be administered BIW or TIW and an Inhibitor Antibody can be administered Q4W. In still another exemplary regimen, a compound of formula I can be administered BIW and an inhibitor described herein can be administered Q2W, Q3W, or Q4W. In some instances, such regimens include administration of an Inhibitor Antibody administered Q2W, Q3W, or Q4W. In yet another exemplary regimen, a compound of formula I can be administered TIW and an inhibitor described herein can be administered Q2W, Q3W, or Q4W. In some instances, such regimens include administration of an Inhibitor Antibody administered Q2W, Q3W, or Q4W. In some instances, such regimens include administration of a compound of formula I administered QD. In some instances, such regimens include administration of a compound of formula I administered QD for at least 21 days. In yet another exemplary regimen, a compound of formula I can be administered QD or QW and an inhibitor (e.g., an Inhibitor Antibody) is administered Q2W, Q3W, or Q4W.

The regimen can be a regimen for administration of an Inhibitor Antibody with a compound of formula I as described herein. In one exemplary regimen including an Inhibitor Antibody, a compound of formula I can be administered BIW or TIW and an Inhibitor Antibody is administered in accordance with the prescribing information provided in, for example, a package insert. In another exemplary regimen, an Inhibitor Antibody is administered at an amount of about 1 mg/kg to about 20 mg/kg on day 1 of the regimen, and Q2W thereafter until disease progression or unacceptable toxicity and a compound of formula I is administered BIW or TIW over the same period of time. In another exemplary regimen, an Inhibitor Antibody is administered at an amount of about 1 mg/kg to about 20 mg/kg on day 1 of a regimen, and Q3W thereafter until disease progression or unacceptable toxicity and a compound of formula I is administered BIW or TIW over the same period of time. An Inhibitor Antibody can be administered Q4W with a compound of formula I, where the compound of formula I is administered, for example, BIW or TIW during the course of such a regimen. An Inhibitor Antibody can be administered Q2W with a compound of formula I, where the compound of formula I is administered, for example, BIW or TIW during the course of such a regimen. In still another exemplary regimen, an Inhibitor Antibody can be administered Q2W or Q4W with a compound of formula I, where the compound of formula I is administered, for example, QD or QW during the course of such a regimen. Such regimens can be repeated as described above (e.g., 1, 2, 3, 4, 5, 6, 7, 8, and 9, 10, 11, 12, or more times).

In another exemplary regimen including an Inhibitor Antibody, a compound of formula I can be administered QD and an Inhibitor Antibody is administered in accordance with the prescribing information provided in, for example, a package insert. In another exemplary regimen, an Inhibitor Antibody is administered at an amount of about 1 mg/kg to about 20 mg/kg on day 1 of the regimen, and Q2W thereafter until disease progression or unacceptable toxicity and a compound of formula I is administered QD over the same period of time. In another exemplary regimen, an Inhibitor Antibody is administered at an amount of about 1 mg/kg to about 20 mg/kg on day 1 of a regimen, and Q3W thereafter until disease progression or unacceptable toxicity and a compound of formula I is administered QD over the same period of time. An Inhibitor Antibody can be administered Q4W with a compound of formula I, where the compound of formula I is administered QD during the course of such a regimen. An Inhibitor Antibody can be administered Q2W with a compound of formula I, where the compound of formula I is administered QD during the course of such a regimen. Such regimens can be repeated as described above (e.g., 1, 2, 3, 4, 5, 6, 7, 8, 9, 10, 11, 12, or more times).

The combinations described herein for treating cancer can be coadministered with other active agents other than those present in the combinations described herein (e.g., anti-cancer agents). Regimens for administration of a combination described herein, including the exemplary regimens set forth above, can be modified as necessary to include administration of such active agents. Administration of such active agents, e.g., anti-cancer agents, can be performed QD, QW, QM, BID, BIW, TIW, Q2W, Q3W, or Q4W, or in accordance with prescribing information for such anti-cancer agents as set forth, for example, in a package insert. Exemplary anti-cancer agents include but are not limited to: ABRAXANE; abiraterone; ace-11; aclarubicin; acivicin; acodazole hydrochloride; acronine; actinomycin; acylfulvene; adecypenol; adozelesin; adriamycin; aldesleukin; all trans-retinoic acid (ATRA); altretamine; ambamustine; ambomycin; ametantrone acetate; amidox; amifostine; aminoglutethimide; aminolevulinic acid; amrubicin; amsacrine; anagrelide; anastrozole; andrographolide; antarelix; anthramycin; aphidicolin glycinate; apurinic acid; ara-CDP-DL-PTBA; arginine deaminase; ARRY-162; ARRY-300; ARRY-142266; AS703026; asparaginase; asperlin; asulacrine; atamestane; atrimustine; axinastatin 1; axinastatin 2; axinastatin 3; azasetron; azatoxin; azatyrosine; azacitidine; AZD8330; azetepa; azotomycin; balanol; batimastat; BAY 11-7082; BAY 43-9006; BAY 869766; bendamustine; benzochlorins; benzodepa; benzoylstaurosporine; beta-alethine; betaclamycin B; betulinic acid; b-FGF inhibitor; bicalutamide; bisantrene; bisaziridinylspermine; bisnafide; bisnafide dimesylate; bistratene A; bisantrene hydrochloride; bleomycin; bleomycin sulfate; busulfan; bizelesin; breflate; bortezomib; brequinar sodium; bropirimine; budotitane; buthionine sulfoximine; bryostatin; cactinomycin; calusterone; calcipotriol; calphostin C; camptothecin derivatives; capecitabine; carboxamide-amino-triazole; carboxyamidotriazole; CaRest M3; CARN 700; caracemide; carbetimer; carboplatin; carmustine; carubicin hydrochloride; carzelesin; castanospermine; cecropin B; cedefingol; celecoxib; cetrorelix; chlorins; chloroquinoxaline sulfonamide; cicaprost; chlorambucil; Chlorofusin; cirolemycin; cisplatin; CI-1040; cis-porphyrin; cladribine; clomifene analogues; clotrimazole; collismycin A; collismycin B; combretastatin A4; combretastatin analogue; conagenin; crambescidin 816; crisnatol; crisnatol mesylate; cryptophycin 8; cryptophycin A derivatives; curacin A; cyclopentanthraquinones; cycloplatam; cypemycin; cyclophosphamide; cytarabine; cytarabine ocfosfate; cytolytic factor; cytostatin; dacarbazine; dactinomycin; daunorubicin; daunorubicin hydrochloride; decarbazine; dacliximab; dasatinib; decitabine; dehydrodidemnin B; deslorelin; dexamethasone; dexifosfamide; dexrazoxane; dexverapamil; dexormaplatin; dezaguanine; dezaguanine mesylate; diaziquone; didemnin B; didox; diethylnorspermine; dihydro 5 azacytidine; dihydrotaxol; 9-dioxamycin; diphenyl spiromustine; docosanol; dolasetron; docetaxel; doxorubicin; doxorubicin hydrochloride; doxifluridine; droloxifene; droloxifene citrate; dromostanolone propionate; dronabinol; duazomycin; duocarmycin SA; ebselen; ecomustine; edelfosine; edrecolomab; edatrexate; eflornithine hydrochloride; eflornithine; elemene; emitefur; elsamitrucin; enloplatin; enpromate; epipropidine; epirubicin; epirubicin hydrochloride; epristeride; erbulozole; eribulin; esorubicin hydrochloride; estramustine; estramustine phosphate sodium; etanidazole; etoposide; etoposide phosphate; etoprine; exemestane; fadrozole; fadrozole hydrochloride; fazarabine; fenretinide; filgrastim; finasteride; flavopiridol; flezelastine; fluasterone; floxuridine; fludarabine phosphate; fludarabine; fluorodaunorubicin hydrochloride; forfenimex; formestane; fluorouracil; floxouridine; flurocitabine; fosquidone; fostriecin sodium; fostriecin; fotemustine; gadolinium texaphyrin; gallium nitrate; galocitabine; ganirelix; gelatinase inhibitors; gemcitabine; geldanamycin; gossyphol; GDC-0973; GSK1120212/trametinib; herceptin; hydroxyurea; hepsulfam; heregulin; hexamethylene bisacetamide; hypericin; ibandronic acid; ibrutinib; idarubicin; idarubicin hydrochloride; ifosfamide; canfosfamide; ilmofosine; iproplatin; idoxifene; idramantone; ilmofosine; ilomastat; imidazoacridones; imatinib (e.g., GLEEVEC); imiquimod; iobenguane; iododoxorubicin; ipomeanol; irinotecan; irinotecan hydrochloride; irsogladine; isobengazole; isohomohalicondrin B; itasetron; iimofosine; interleukin Il (including recombinant interleukin IL-2; or r1L₂); interferon alfa-2a; interferon alfa-2b; interferon alfa-n1; interferon alfa-n3; interferon beta-1a; interferon gamma-1b; jasplakinolide; kahalalide F; lamellarin N triacetate; lanreotide; leinamycin; lenograstim; lentinan sulfate; leptolstatin; letrozole; leuprorelin; levamisole; liarozole; lissoclinamide 7; lobaplatin; lombricine; lometrexol; lonidamine; losoxantrone; lovastatin; loxoribine; lurtotecan; lutetium texaphyrin; lysofylline; lanreotide acetate; lapatinib; letrozole; leucovorin; leuprolide acetate; liarozole hydrochloride; lometrexol sodium; lomustine; lenalidomide; lenvatinib; losoxantrone hydrochloride; LY294002; pomalidomide; maitansine; mannostatin A; marimastat; masoprocol; maspin; matrilysin inhibitors; menogaril; merbarone; meterelin; methioninase; metoclopramide; MIF inhibitor; mifepristone; miltefosine; mirimostim; mitoguazone; mitolactol; mitonafide; mitoxantrone; mofarotene; molgramostim; mopidamol; mycaperoxide B; myriaporone; maytansine; mechlorethamine hydrochloride; megestrol acetate; melengestrol acetate; melphalan; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nafarelin; nagrestip; napavin; naphterpin; nartograstim; nedaplatin; nemorubicin; neridronic acid; nilutamide; nisamycin; nitric oxide modulators; nitroxide antioxidant; nitrullyn; nocodazole; nogalamycin; oblimersen (GENASENSE); octreotide; okicenone; oligonucleotides; onapristone; ondansetron; ondansetron; oracin; oral cytokine inducer; ormaplatin; oxisuran; oxaloplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; porfiromycin; prednisone; prostaglandin J2; pyrazoloacridine; paclitaxel; PD035901; PD184352; PD318026; PD98059; peliomycin; pentamustine; peplomycin sulfate; PKC412; pipobroman; piposulfan; piroxantrone hydrochloride; plicamycin; plomestane; podophyllotoxin; polyphenol E; porfimer sodium; porfiromycin; prednimustine; procarbazine; procarbazine hydrochloride; puromycin; puromycin hydrochloride; pyrazofurin; raltitrexed; ramosetron; retelliptine demethylated; rhizoxin; rituximab; RII retinamide; rogletimide; rohitukine; romurtide; roquinimex; rubiginone B 1; ruboxyl; riboprine; romidepsin; safingol; safingol hydrochloride; saintopin; sarcophytol A; sargramostim; semustine; sizofiran; sobuzoxane; sodium borocaptate; sodium phenylacetate; solverol; sonermin; sorafenib; sunitinib; sparfosic acid; spicamycin D; spiromustine; splenopentin; spongistatin 1; Spongistatin 2; Spongistatin 3; Spongistatin 4; Spongistatin 5; Spongistatin 6; Spongistatin 7; Spongistatin 8; and Spongistatin 9; squalamine; stipiamide; stromelysin inhibitors; sulfinosine; suradista; suramin; swainsonine; SB239063; selumetinib/AZD6244; simtrazene; SP600125; sparfosate sodium; sparsomycin; spirogermanium hydrochloride; spiroplatin; streptonigrin; streptozocin; sulofenur; tallimustine; tamoxifen methiodide; tauromustine; tazarotene; tecogalan sodium; tegafur; tellurapyrylium; temoporfin; temozolomide; teniposide; tetrachlorodecaoxide; tetrazomine; thaliblastine; thiocoraline; thrombopoietin; thymalfasin; thymopoietin receptor agonist; thymotrinan; tirapazamine; titanocene bichloride; topsentin; toremifene; tretinoin; triacetyluridine; triciribine; trimetrexate; triptorelin; tropisetron; turosteride; tyrphostins; talisomycin; TAK-733; taxotere; tegafur; teloxantrone hydrochloride; teroxirone; testolactone; thiamiprine; thioguanine; thiotepa; tiazofurin; tirapazamine; toremifene citrate; trastuzumab; trestolone acetate; triciribine phosphate; trimetrexate; trimetrexate glucuronate; triptorelin; tubulozole hydrochloride; tumor necrosis factor-related apoptosis-inducing ligand (TRAIL); UBC inhibitors; ubenimex; U0126; uracil mustard; uredepa; vapreotide; variolin B; velaresol; veramine; verteporfin; vinorelbine; vinxaltine; vitaxin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; wortmannin; XL518; zanoterone; zeniplatin; zilascorb; zinostatin stimalamer; zinostatin; and zorubicin hydrochloride.

Other exemplary anti-cancer agents include Erbulozole (e.g., R-55104); Dolastatin 10 (e.g., DLS-10 and NSC-376128); Mivobulin isethionate (e.g., CI-980); NSC-639829; Discodermolide (e.g., NVP-XX-A-296); ABT-751 (Abbott; e.g., E-7010); Altorhyrtin A; Altorhyrtin C); Cemadotin hydrochloride (e.g., LU-103793 and NSC-D-669356); Epothilone A; Epothilone B; Epothilone C; Epothilone D; Epothilone E; Epothilone F; Epothilone B N-oxide; Epothilone A N-oxide; 16-aza-epothilone B; 21-aminoepothilone B; 21-hydroxyepothilone D; 26-fluoroepothilone; Auristatin PE (e.g., NSC-654663); Soblidotin (e.g., TZT-1027); LS-4559-P (Pharmacia; e.g., LS-4577); LS-4578 (Pharmacia; e.g., LS-477-P); LS-4477 (Pharmacia); LS-4559 (Pharmacia); RPR-112378 (Aventis); DZ-3358 (Daiichi); FR-182877 (Fujisawa; e.g., WS-9265B); GS-164 (Takeda); GS-198 (Takeda); KAR-2 (Hungarian Academy of Sciences); B SF-223651 (BASF; e.g., ILX-651 and LU-223651); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (e.g., LY-355703); AC-7739 (Ajinomoto; e.g., AVE-8063A and CS-39-HCl); AC-7700 (Ajinomoto; e.g., AVE-8062; AVE-8062A; CS-39-L-Ser'HCl; and RPR-258062A); Vitilevuamide; Tubulysin A; Canadensol; CA-170 (Curis, Inc.); Centaureidin (e.g., NSC-106969); T-138067 (Tularik; e.g., T-67; TL-138067 and TI-138067); COBRA-1 (Parker Hughes Institute; e.g., DDE-261 and WHI-261); H10 (Kansas State University); H16 (Kansas State University); Oncocidin Al (e.g., BTO-956 and DIME); DDE-313 (Parker Hughes Institute); Fijianolide B; Laulimalide; SPA-2 (Parker Hughes Institute); SPA-1 (Parker Hughes Institute; e.g., SPIKET-P); 3-IAABU (Cytoskeleton/Mt. Sinai School of Medicine; e.g., MF-569); Narcosine (e.g., NSC-5366); Nascapine; D-24851 (Asta Medica); A-105972 (Abbott); Hemiasterlin; 3-BAABU (Cytoskeleton/Mt. Sinai School of Medicine; e.g., MF-191); TMPN (Arizona State University); Vanadocene acetylacetonate; T-138026 (Tularik); Monsatrol; Indanocine (i.e., NSC-698666); 3-IAABE (Cytoskeleton/Mt. Sinai School of Medicine); A-204197 (Abbott); T-607 (Tuiarik; e.g., T-900607); RPR-115781 (Aventis); Eleutherobins (e.g., Desmethyleleutherobin; Desaetyleleutherobin; Isoeleutherobin A; and Z-Eleutherobin); Caribaeoside; Caribaeolin; Halichondrin B; D-64131 (Asta Medica); D-68144 (Asta Medica); Diazonamide A; A-293620 (Abbott); NPI-2350 (Nereus); Taccalonolide A; TUB-245 (Aventis); A-259754 (Abbott); Diozostatin; (−)-Phenylahistin (e.g., NSCL-96F037); D-62638 (Asta Medica); D-62636 (Asta Medica); Myoseverin B; D-43411 (Zentaris; e.g., D-81862); A-289099 (Abbott); A-318315 (Abbott); HTI-286 (e.g., SPA-110; trifluoroacetate salt) (Wyeth); D-82317 (Zentaris); D-82318 (Zentaris); SC-12983 (NCI); Resverastatin phosphate sodium; BPR-OY-007 (National Health Research Institutes); and SSR-250411 (Sanofi)); goserelin; leuprolide; triptolide; homoharringtonine; topotecan; itraconazole; deoxyadenosine; sertraline; pitavastatin; clofazimine; 5-nonyloxytryptamine; vemurafenib; dabrafenib; gefitinib (IRESSA); erlotinib (TARCEVA); cetuximab (ERBITUX); lapatinib (TYKERB); panitumumab (VECTIBIX); vandetanib (CAPRELSA); afatinib/BIBW2992; CI-1033/canertinib; neratinib/HKI-272; CP-724714; TAK-285; AST-1306; ARRY334543; ARRY-380; AG-1478; dacomitinib/PF299804; OSI-420/desmethyl erlotinib; AZD8931; AEE726; pelitinib/EKB-569; CUDC-101; WZ8040; WZ4002; WZ3146; AG-490; XL647; PD153035; 5-azathioprine; 5-aza-2′-deoxycytidine; 17-N-Allylamino-17-Demethoxygeldanamycin (17-AAG); 20-epi-1,25-dihydroxy vitamin D3; 5-ethynyluracil; and BMS-599626.

In some embodiments, the combinations described herein are coadministered with an anti-cancer agent described above, where the anti-cancer agent has known activity against a particular cancer (e.g., gemcitabine coadministered with a combination described herein for treating pancreatic cancer). The anti-cancer agents above can be approved for use in treating certain indications (e.g., certain cancers) at concentrations, amounts, and using treatment regimens known in the art.

It is understood that modifications which do not substantially affect the activity of the various embodiments of this invention are also included within the definition of the invention provided herein. Although the invention has been described with reference to the disclosed embodiments, those skilled in the art will readily appreciate that the specific examples and studies detailed above are only illustrative of the invention. It should be understood that various modifications can be made without departing from the spirit of the invention.

### EXAMPLES

The following examples are included for illustrative purposes only and are not intended to limit the scope of the invention.

The present example, was a Phase Ib/2 open-label dose finding study for HBI-8000 (also referred to in the art as “chidamide” or “tucidinostat” and also referred to herein as a compound of formula I) in combination with nivolumab in patients with advanced solid tumors, including melanoma, RCC (renal cell carcinoma), and NSCLC (non-small cell lung cancer). This study was conducted in accordance with Good Clinical Practice guidelines and the Declaration of Helsinki and with assurance/approval from the US Department of Health and Human Services; the protocol was approved by the institutional review boards or ethics committees of all participating sites. All patients provided written informed consent to participate before enrollment.

One of the objectives of Phase Ib was to evaluate the safety and tolerability of HBI-8000 when combined with a standard dose and regimen of nivolumab, determine the maximum tolerated dose (MTD), and/or a recommended phase II dose (RP2D) for HBI-8000, and to evaluate the frequency and severity of toxicities of the combination assessed by NCI CTC version 4.03. Another objective included assessing the PK (pharmacokinetics) of HBI-8000, and the effect of HBI-8000 on the electrocardiogram (ECG) QT interval corrected for heart rate (QTc interval) if any.

HBI-8000 at three dose levels (20 mg, 30 mg, and 40 mg) given orally twice a week (BIW) were evaluated. Doses were taken approximate 30 minutes after a meal. The starting dose (20 mg) was selected based on pharmacokinetic profiles established for monotherapy in Japanese patients with T-cell lymphoma. Doses up to 40 mg BIW continuous dosing administered as a single agent in heavily pre-treated lymphoma patients was safe and toxicities were manageable. In combination with nivolumab, because the monoclonal antibody is not likely to interfere with HBI-8000 metabolism, 20 mg was expected to be safe as the starting dose in combination with the standard dose of nivolumab.

The decision to escalate the dose was based on the conventional 3+3 design and observed incidences of dose-limiting toxicities (DLTs). Each treatment cycle was defined as 28 days. To be evaluable for DLT, a subject must have developed DLT, or received the minimum 75% of the planned HBI-8000 dose within the 28-day DLT evaluation period in the absence of treatment delays due to toxicities. Subjects experiencing DLT within the first two weeks after having received 100% of assigned dose of HBI-8000 are also evaluable for DLT. Unacceptable toxicities were defined as grade 3 or higher non-hematologic and hematologic toxicities with clinical complications. Unacceptable toxicities observed over the first 28 days following HBI-8000 administration were considered as DLTs. The RP2D was identified as the highest dose at which the DLT incidence was less than 33.3%. HBI-8000 administration continued until disease progression or unacceptable toxicity was observed. The efficacy of the combination was further evaluated in an expanded cohort of selected patient populations at RP2D based on an objective response rate according to Response Evaluation Criteria in Solid Tumors (RECIST) version 1.1 criteria, progression-free survival, and duration of responses.

### Patient Selection

Adults 18 years of age or older with histopathologically or cytologically confirmed advanced non-uveal melanoma, RCC, or NSCLC for whom the use of nivolumab is indicated were eligible for screening, provided the use of nivolumab was indicated. Patients had to have ≥1 measurable target lesion as defined by RECIST v.1.1 (Eisenhauer 2009), an Eastern Cooperative Oncology Group (ECOG) performance status ≤1 (Oken 1982), and life expectancy of at least 12 weeks. Major exclusion criteria included hypersensitivity to monoclonal antibodies, cardiovascular illness, uncontrolled hypertension, active brain metastasis, leptomeningeal disease, severe gastrointestinal disease, autoimmune disease, severe infection, human immunodeficiency virus, and active hepatitis B.

### Assessments

Safety evaluations included physical examinations, vital signs, ECGs, ECOG performance status, and laboratory tests. Adverse events (AEs) were graded according to the National Cancer Institute Common Terminology Criteria for Adverse Events (NCI CTCAE) Version 4.03 and classified using Medical Dictionary for Regulatory Activities (MedDRA) classification system version 18.0 or higher.

Tumor assessments were performed every 8 weeks until treatment discontinuation, then every 12 (±1) weeks in subjects in whom disease progression has not yet been observed, and followed the guidelines specified by RECIST v.1.1 and immune-related Response Evaluation Criteria In Solid Tumors (iRECIST).

### Statistical Analyses

Statistical analyses of safety, tolerability, and anti-tumor activity were primarily descriptive in nature. Toxicities were tabulated by type and grade. The safety population included all patients who received ≥1 dose of HBI-8000 and nivolumab. Continuous variables were summarized using descriptive statistics and categorical variables were summarized with number and percentage at each category. Pharmacokinetic parameters were derived using noncompartmental methods with Phoenix® WinNonlin® Version 6.4 (Certara, L. P., Princeton, NJ, USA).

### Non-Compartmental PK Analysis

Non-compartmental PK analysis for HBI-8000 was conducted in Phase 1b using serial plasma concentration data collected up to 24 hours post-dose for the first (Cycle 1 Day 1; C1D1) dose and up to 7 hours post-dose for the ninth (C2D1) dose of HBI-8000. In conjunction with PK, Holter monitors were used to record 12-lead continuous digital ECGs (under controlled conditions to minimize digital noise) on a baseline day (within 1-7 days prior to C1D1) and on C1D1 (Phase 1b only). Triplicate 10-second ECGs were extracted from the Holter flashcard at matched time-points on the baseline day and on C1D1 up to 4 hours post-dose and the PR, QRS, RR, and QT intervals were analyzed by a Central ECG Laboratory. Meal-times (breakfast) were standardized across both days and occurred after or ≥1 hour before the scheduled extraction times for triplicate ECGs. The measured QT data were corrected for heart rate using Fridericia (QTcF).

### Results

Seventeen patients were enrolled into Phase 1b: 3 at 20 mg, 7 at 30 mg, and 7 at the 40 mg dose level from August 2016 to August 2017. The patient characteristics are summarized in Table 1. Other than the underlying cancer diagnosis, there was no apparent difference in baseline characteristics affecting safety assessment.

### Patients

### Demographic and Other Baseline Characteristics

TABLE 1
Patient Characteristics by Dose Cohort

| | | 20 mg | 30 mg | 40 mg | All | |
|---|---|---|---|---|---|---|
| | | (N = 3) | (N = 7) | (N = 7) | (N = 17) | |
| Characteristics | Value | n (%) | n (%) | n (%) | n (%) | |
| | n | 3 | 7 | 7 | 17 | |
| Age, years | median (range) | 69.0 (52-69) | 71.0 (52-79) | 72.0 (40-87) | 70.0 | (40-87) |
| | <65 | 1 (33.3%) | 3 (42.9%) | 1 (14.3%) | 5 | (29.4%) |
| | ≥65 | 2 (66.7%) | 4 (57.1%) | 6 (85.7%) | 12 | (70.6%) |
| Sex | F | 1 (33.3%) | 1 (14.3%) | 3 (42.9%) | 5 | (29.4%) |
| | M | 2 (66.7%) | 6 (85.7%) | 4 (57.1%) | 12 | (70.6%) |
| ECOG PS | 0 | 1 (33.3%) | 2 (28.6%) | 1 (14.3%) | 4 | (23.5%) |
| | 1 | 2 (66.7%) | 5 (71.4%) | 6 (85.7%) | 13 | (76.5%) |
| Tumor Type | MELANOMA | | 3 (42.9%) | 2 (28.6%) | 5 | (29.4%) |
| | NSCLC | | 1 (14.3%) | 4 (57.1%) | 5 | (29.4%) |
| | RCC-Clear cell | 3 (100.0%) | 3 (42.9%) | 1 (14.3%) | 7 | (41.2%) |
| Previous Surgery | Yes | 3 (100.0%) | 7 (100.0%) | 7 (100.0%) | 17 | (100.0%) |
| Previous Cancer | BIOLOGIC ¹ | 2 (66.7%) | 3 (42.9%) | 1 (14.3%) | 6 | (35.3%) |
| Therapy | | | | | | |
| | BIOLOGIC ¹ | | | 1 (14.3%) | 1 | (5.9%) |
| | TIL ² | | | | | |
| | BIOLOGIC | 1 (33.3%) | | | 1 | (5.9%) |
| | Investigational | | | | | |
| | CHEMOTHERAPY | | | 4 (57.1%) | 4 | (23.5%) |
| | COMBINATION | | 1 (14.3%) | | 1 | (5.9%) |
| | None | | 3 (42.9%) | 1 (14.3%) | 4 | (23.5%) |

Abbreviations: RCC = Renal-cell carcinoma; NSCLC = Non-small Cell Lung Cancer.
¹ Targeted Therapy, immunotherapy excluding anti-PD-1, anti-PD-L1, and Ipilimumab ²
² Tumor Infiltrating Lymphocytes therapy

### Exposure:

Overall, the median (min, max) duration of the study drug exposure was 2.8 (0.6, 28.3) months for HBI 8000 and 2.3 (0.5, 22.5) months for nivolumab. The median (min, max) total number of cycles completed was 3.0 (1, 31) cycles for HBI-8000 and 2.5 (1, 24) cycles for nivolumab.

### Safety

### Dose-Limiting Toxicities (DLT)

A total of 17 patients were enrolled in the study; two patients were not evaluable for DLT; 1 in the 30-mg BIW dose cohort withdrew consent early and 1 in the 40-mg BIW dose cohort discontinued early because of the necessity of steroid at dose higher than protocol permitted to manage an underlying lung disease. Neither patient received the minimum 75% of the HBI-8000 dose within the 28-day DLT evaluation period. They were deemed not evaluable for DLT while being included in overall safety analysis.

For the 15 DLT-evaluable patients, the first 3 were in the 20-mg dose cohort, followed by 3 in the 30-mg dose cohort. None experienced a DLT. Of the 6 patients in the 40-mg cohort, 1 patient with melanoma experienced Grade 3 headache 24-48 hours after receiving the first dose and grade 3 diarrhea after the third dose, and 1 patient with RCC had Grade 3 fatigue on days of HBI-8000 administration, which were considered DLTs. Thus, 40 mg was deemed to have exceeded the MTD. Three additional patients were evaluated at the 30-mg BIW dose level. No DLT was observed. The RP2D for further evaluation of the safety and efficacy of HBI-8000 in combination with nivolumab was determined to be 30 mg BIW.

### Treatment-Emergent Adverse Events (TEAE)

All 17 patients who received HBI-8000 also received nivolumab. They were included in the safety analysis. The mean duration of HBI-8000 treatment was 6.2 months. A detailed overview of the TEAEs and treatment modification attributed to HB-8000 alone, nivolumab alone, or both is provided in Table 2.

TABLE 2
Summary of Number of Subjects Experienced Treatment-
Emergent Adverse Events (TEAEs) by Dose Levels

| | 20 mg | | 30 mg | | 40 mg | | Total | |
|---|---|---|---|---|---|---|---|---|
| | N = 3 | | N = 7* | | N = 7* | | N = 17 | |
| Description | n (%) | | n (%) | | n (%) | | n (%) | |
| Any | 3 | (100.0%) | 7 | (100.0%) | 7 | (100.0%) | 17 | (100.0%) |
| TEAE related to HBI-8000 alone | 0 | (0%) | 4 | (57.1%) | 6 | (85.7%) | 10 | (58.8%) |
| TEAE related to Nivolumab alone | 2 | (66.7%) | 6 | (85.7%) | 2 | (28.6%) | 10 | (58.8%) |
| TEAE related to HBI-8000 & Nivolumab | 3 | (100.0%) | 5 | (71.4%) | 3 | (42.9%) | 11 | (64.7%) |
| Any SAE | 1 | (33.3%) | 6 | (85.7%) | 4 | (57.1%) | 11 | (64.7%) |
| SAE related to HBI-8000 alone | 0 | (0%) | 0 | (0%) | 0 | (0%) | 0 | (0%) |
| SAE related to Nivolumab alone | 1 | (33.3%) | 2 | (28.6%) | 1 | (14.3%) | 4 | (23.5%) |
| SAE related to HBI-8000 & Nivolumab | 0 | (0%) | 0 | (0%) | 0 | (0%) | 0 | (0%) |
| TEAE resulting in discontinuation of | 2 | (66.7%) | 1 | (14.3%) | 2 | (28.6%) | 5 | (29.4%) |
| HBI-8000 alone | | | | | | | | |
| TEAE resulting in discontinuation of | 1 | (33.3%) | 4 | (57.1%) | 2 | (28.6%) | 7 | (41.2%) |
| Nivo alone | | | | | | | | |
| TEAE resulting in discontinuation of | 0 | (0%) | 1 | (14.3%) | 2 | (28.6%) | 3 | (17.6%) |
| both HBI-8000 and Nivo | | | | | | | | |

*1 subject was not evaluable for DLT

All 17 patients experienced TEALs; not all TEALs were related to HBI-8000, nivolumab or both. TEALs related to both HBI-8000 and nivolumab combination were observed in 11 (64.7%) subjects while those related to HBI-8000 or nivolumab alone were observed in 10 (58.8%) subjects. Six subjects experienced TEALs attributed to the combination and TEALs associated with HBI-8000 alone. The incidence of TEAL related to HBI-8000 alone appeared to increase with escalating HBI-8000 dose. However, a similar trend was not observed with TEAEs associated with the combination or nivolumab alone. Despite any differences in incidence, TEAEs resulting in treatment discontinuation appeared similar among the three dose levels.

Two patients experienced only Grade 1 and 2 TEAEs while 15 experienced Grade 3 or higher TEAEs. It was also noted that TEAEs related to HBI-8000, nivolumab, alone or in combination, were the minority. Furthermore Grade 3 TEAEs regardless causality were infrequent (Table 3 A-C).

The TEAEs observed in ≥40% of all subjects were fatigue, decreased appetite, dyspnea, platelet count decreased, anemia, weight loss, nausea, diarrhea, and peripheral edema. Although it seems more TEAEs were reported at HBI-8000 30 mg and 40 mg when compared to 20 mg, there was no apparent increase of Grade ≥3 events at 40 mg. When TEAEs associated with HBI-8000 regardless of severity were examined, there seems to be a slight trend of increase at higher dose. However, this trend was not detected when TEAEs related to HBI-8000 and nivolumab combination were examined.

The Grade ≥3 TEAEs observed in ≥15% of patients were fatigue, hypophosphatemia, decreased lymphocyte count, increased lipase levels, and hypoxia. Except for fatigue and hypoxia, they were asymptomatic. Grade ≥3 TEAEs occurred in all HBI-8000 dose cohorts.

SAEs were observed in 11 patients, 4 experienced SAE related to nivolumab alone. No SAE was attributed to HBI-8000 alone or HBI-8000 and nivolumab combination. TEAE led to discontinuation of both HBI-8000 and nivolumab were observed in 3 patients (17.6%). One patient died of TEAE unrelated to HBI-8000 or nivolumab. No clear differences in the incidence, severity, causality, or seriousness of TEAEs were detected at the dose levels tested.

TABLE 3A
Description of Adverse Events Observed in ≥4
Patients Administered 20 mg of HBI-8000

| | 20 mg | | | | |
|---|---|---|---|---|---|
| | (N = 3) | | | | |
| | | | Related | | |
| Adverse Event, n (%) | All | | HBI | Nivo | Both |
| Total N = 17 | G < 3 | G ≥ 3 | Any G | Any G | Any G |
| Fatigue | 2 (67) | 1 (33) | | | 1 (33) |
| Decreased appetite | 1 (33) | | | | |
| Dyspnoea | 2 (67) | | | | 1 (33) |
| Anaemia | | | | | |
| Nausea | 2 (67) | 1 (33) | | | |
| Diarrhoea | 2 (67) | | | 1 (33) | 1 (33) |
| Oedema peripheral | 1 (33) | | | | |
| Platelet count decreased | | | | | |
| Weight decreased | 1 (33) | | | 1 (33) | |
| Hypocalcaemia | 1 (33) | 1 (33) | | | |
| Dehydration | 1 (33) | | | | |
| Dizziness | 1 (33) | | | | |
| Hypokalaemia | | 1 (33) | | | |
| Hypophosphataemia | | | | | |
| Hypotension | | | | | |
| Lipase increased | | | | | |
| Lymphocyte count decreased | | | | | |
| Pleural effusion | | | | | |
| White blood cell count decreased | | | | | |
| Alanine aminotransferase increased | 1 (33) | | | | 1 (33) |
| Aspartate aminotransferase increased | 1 (33) | | | | 1 (33) |
| Atrial fibrillation | | | | | |
| Back pain | | | | | |
| Contusion | | | | | |
| Fall | | | | | |
| Headache | 1 (33) | | | | |
| Hypoxia | | | | | |
| Neutrophil count decreased | | | | | |
| Non-cardiac chest pain | | | | | |
| Vomiting | 1 (33) | | | | |

TABLE 3B
Description of Adverse Events Observed in ≥4
Patients Administered 30 mg of HBI-8000

| | 30 mg | | | | |
|---|---|---|---|---|---|
| | (N = 7*) | | | | |
| | | | Related | | |
| | All | | HBI | Nivo | Both |
| Adverse Event, n (%) | G < 3 | G ≥ 3 | Any G | Any G | Any G |
| Fatigue | 5 (71) | 1 (14) | | | 4 (57) |
| Decreased appetite | 4 (57) | 1 (14) | 1 (14) | | 3 (43) |
| Dyspnoea | 4 (57) | | | 1 (14) | 1 (14) |
| Anaemia | 4 (57) | 1 (14) | | | 3 (43) |
| Nausea | 3 (43) | | 1 (14) | | |
| Diarrhoea | 3 (43) | | | | 3 (43) |
| Oedema peripheral | 4 (57) | 1 (14) | 1 (14) | | 1 (14) |
| Platelet count decreased | 4 (57) | | 1 (14) | | 3 (43) |
| Weight decreased | 5 (71) | | | | 3 (43) |
| Hypocalcaemia | 3 (43) | | | | 2 (29) |
| Dehydration | 2 (29) | 1 (14) | | | 1 (14) |
| Dizziness | 2 (29) | | 1 (14) | | 1 (14) |
| Hypokalaemia | 2 (29) | 1 (14) | | | 1 (14) |
| Hypophosphataemia | 3 (43) | 3 (43) | 1 (14) | | 2 (29) |
| Hypotension | 1 (14) | 2 (29) | | | 1 (14) |
| Lipase increased | 3 (43) | 4 (57) | | 1 (14) | 2 (29) |
| Lymphocyte count decreased | 3 (43) | 3 (43) | | | 2 (29) |
| Pleural effusion | 3 (43) | | | | 1 (14) |
| White blood cell count decreased | 2 (29) | 1 (14) | 2 (29) | | 2 (29) |
| Alanine aminotransferase increased | 2 (29) | | | | 2 (29) |
| Aspartate aminotransferase increased | 2 (29) | | | | 2 (29) |
| Atrial fibrillation | 1 (14) | | | | |
| Back pain | 4 (57) | | | 2 (29) | 1 (14) |
| Contusion | 1 (14) | | | | 1 (14) |
| Fall | 2 (29) | | | 1 (14) | 1 (14) |
| Headache | 2 (29) | | | | 1 (14) |
| Hypoxia | 1 (14) | 3 (43) | | 1 (14) | 1 (14) |
| Neutrophil count decreased | 2 (29) | 1 (14) | 1 (14) | | 1 (14) |
| Non-cardiac chest pain | 2 (29) | | | | |
| Vomiting | 1 (14) | | | | 1 (14) |

TABLE 3C
Description of Adverse Events Observed in ≥4 Patients Administered 40
mg of HBI-8000. Table 3C also tallies the adverse events observed in the
total number of patients administered 20 mg, 30 mg and 40 mg of HBI-8000.

| | 40 mg | | | | | |
|---|---|---|---|---|---|---|
| | (N = 7*) | | | | | |
| | | | Related | | | |
| | All | | HBI | Nivo | Both | Total |
| Adverse Event, n (%) | G < 3 | G ≥ 3 | Any G | Any G | Any G | (N = 17) |
| Fatigue | 2 (29) | 2 (29) | 1 (14) | | 2 (29) | 10 (59) |
| Decreased appetite | 3 (43) | 1 (14) | 2 (29) | | 1 (14) | 9 (53) |
| Dyspnoea | 3 (43) | 1 (14) | | | 1 (14) | 9 (53) |
| Anaemia | 4 (57) | 1 (14) | 2 (29) | | 1 (14) | 8 (47) |
| Nausea | 3 (43) | | 2 (29) | | 2 (29) | 8 (47) |
| Diarrhoea | 2 (29) | 1 (14) | 1 (14) | 1 (14) | 2 (29) | 7 (41) |
| Oedema peripheral | 2 (29) | | | | 1 (14) | 7 (41) |
| Platelet count decreased | 3 (43) | | 2 (29) | | 1 (14) | 7 (41) |
| Weight decreased | 1 (14) | | | | 1 (14) | 7 (41) |
| Hypocalcaemia | 2 (29) | | 1 (14) | | 1 (14) | 6 (35) |
| Dehydration | 2 (29) | | | | | 5 (29) |
| Dizziness | 2 (29) | | 1 (14) | | 1 (14) | 5 (29) |
| Hypokalemia | 1 (14) | | | | 1 (14) | 5 (29) |
| Hypophosphataemia | 2 (29) | | 1 (14) | | 1 (14) | 5 (29) |
| Hypotension | 3 (43) | | | | 1 (14) | 5 (29) |
| Lipase increased | 1 (14) | | | | 1 (14) | 5 (29) |
| Lymphocyte count decreased | 2 (29) | | | | 2 (29) | 5 (29) |
| Pleural effusion | 2 (29) | | | | | 5 (29) |
| White blood cell count decreased | 3 (43) | | 2 (29) | | 1 (14) | 5 (29) |
| Alanine aminotransferase increased | 1 (14) | | | | 1 (14) | 4 (24) |
| Aspartate aminotransferase increased | 1 (14) | | | | 1 (14) | 4 (24) |
| Atrial fibrillation | 2 (29) | 1 (14) | | | 1 (14) | 4 (24) |
| Back pain | | | | | | 4 (24) |
| Contusion | 3 (43) | | 1 (14) | | 2 (29) | 4 (24) |
| Fall | 2 (29) | | | | | 4 (24) |
| Headache | 1 (14) | 1 (14) | 1 (14) | | 1 (14) | 4 (24) |
| Hypoxia | | | | | | 4 (24) |
| Neutrophil count decreased | 2 (29) | 1 (14) | 1 (14) | | 1 (14) | 4 (24) |
| Non-cardiac chest pain | 2 (29) | | 1 (14) | | | 4 (24) |
| Vomiting | 2 (29) | | | | 2 (29) | 4 (24) |

*1 subject was not evaluable for DLT.

### Pharmacokinetics

### HBI-8000 Concentrations

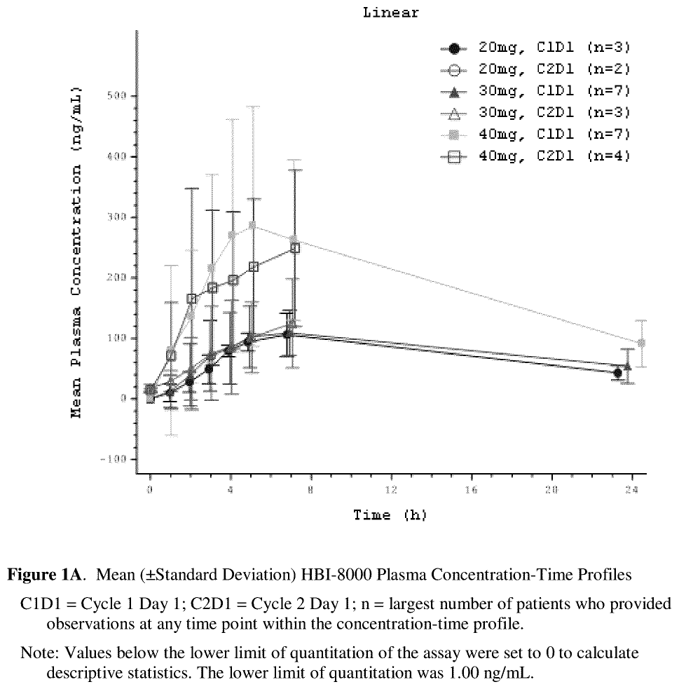

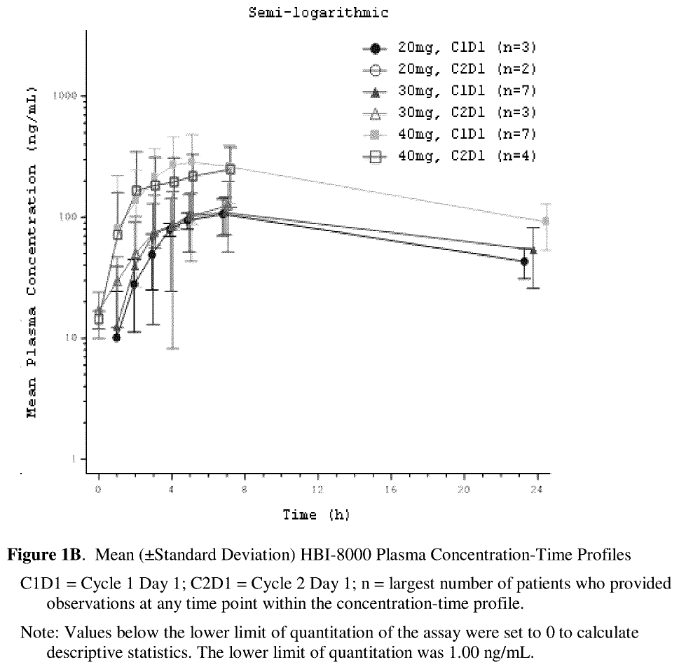

Following a single dose of HBI-8000 alone in Cycle 1 Day 1 (C1D1), the mean plasma concentrations of HBI-8000 were generally comparable between the 20 mg and 30 mg doses. Mean HBI-8000 concentrations were higher for the 40 mg compared to the 30 mg dose at every sampling time-point up to 24 hours after dosing. Similar trends were observed for HBI 8000 concentrations up to 7 hours after dosing for C2D1 following continuous dosing on BIW schedule for 4 weeks in Cycle 1. Of note, nivolumab was administered after plasma collection for HBI-8000 PK determination for the first and the ninth doses of HBI-8000 were concluded. See FIG. 1A-B.

### HBI-8000 Pharmacokinetic Parameters

In the 20, 30, and 40 mg BIW dose cohorts, the median time to peak plasma concentration (tmax) ranged from 5 to 7 hours post dose (Table 4). Exposure parameters [Cmax, AUC₀₋₂₄, and/or AUC₀₋₇] were generally comparable between the 20 and 30 mg doses at C1D1. An increase in HBI-8000 exposure was observed for the 40 mg dose relative to the 30 mg dose, for both the first dose (C1D1) and the ninth dose (C2D1).

No significant accumulation of HBI-8000 was observed when comparing the pharmacokinetic parameters between the first and ninth doses. The geometric mean ratios (C2D1:C1D1) of the HBI-8000 exposure parameters ranged from 1.08 to 1.18 for Cmax and 1.36 to 1.37 for AUC₀₋₇ across the 30- and 40-mg BIW dosing regimens.

TABLE 4
Geometric mean (geometric CV %) for HBI-8000 pharmacokinetic parameters on Day 1 of Cycles 1 and 2

| | C1D1 | | | | | | C2D1 | | | | |
|---|---|---|---|---|---|---|---|---|---|---|---|
| | 20 mg | | 30 mg | | 40 mg | | 20 mg | 30 mg | | 40 mg | |
| Parameter (unit) | (n = 3) | | (n = 7) | | (n = 7) | | (n = 2)^{b} | (n = 3) | | (n = 4) | |
| Cₘₐₓ (ng/mL) | 105 | (32) | 118 | (34) | 259 | (74) | 83.1, 182 | 113 | (94) | 229 | (73) |
| Cₘₐₓ/D (ng/mL/mg) | 5.24 | (32) | 3.92 | (34) | 6.49 | (74) | 4.16, 9.10 | 3.76 | (94) | 5.73 | (73) |
| tₘₐₓ (h)a | 6.6 | (5.0, 6.7) | 6.5 | (3.1, 26) | 4.8 | (1.0, 6.6) | 3.0, 6.7 | 6.7 | (4.1, 6.8) | 6.8 | (2.0, 6.9) |
| AUC₀₋₂₄ (ng · h/mL) | 1590 | (30) | 1734 | (29) | 3690 | (55) | n/a | n/a | | n/a | |
| AUC₀₋₂₄/D (ng · h/mL/mg) | 79.5 | (30) | 57.8 | (29) | 92.2 | (55) | n/a | n/a | | n/a | |
| AUC₀₋₇ (ng · h/mL) | 384 | (9) | 342 | (98) | 973 | (124)^{c} | 250, 728 | 392 | (117) | 929 | (89) |
| AUC₀₋₇/D (ng · h/mL/mg) | 19.2 | (9) | 11.4 | (98) | 24.3 | (124)^{c} | 12.5, 36.4 | 13.1 | (117) | 23.2 | (89) |

| | | | | | | | | |
|---|---|---|---|---|---|---|---|---|
| C_{Pre} (ng/mL) | n/a | n/a | n/a | 4.76, 4.79 | 16.1 | (44) | 14.2 | (17) |
| C_{Pre}/D (ng/mL/mg) | n/a | n/a | n/a | 0.238, 0.240 | 0.536 | (44) | 0.356 | (17) |
| Cₘₐₓ Ratio (C2D1/C1D1) | n/a | n/a | n/a | 1.06, 1.80 | 1.08 | (42) | 1.18 | (33) |
| AUC₀₋₇ Ratio | n/a | n/a | n/a | 0.69, 1.99 | 1.37 | (13) | 1.36 | (63)^{d} |
| (C2D1/C1D1) | | | | | | | | |

Abbreviations: AUC₀₋₇ = area under the concentration-time curve (AUC) from zero to 7 hours post dose; AUC₀₋₂₄ = AUC from zero to 24 hours post dose; Cₘₐₓ = maximum observed concentration; C_{Pre} = pre-dose (trough) concentration; CV % = percent coefficient of variation; /D = dose-normalized parameter; n = number of valid observations for each parameter, unless otherwise stated; n/a: not applicable; tₘₐₓ = time to Cₘₐₓ.
^{a}Data presented for this parameter are median (minimum, maximum).
^{b}Individual values are presented since n = 2.
^{c}n = 6
^{d}n = 3.

Analysis of QTcF with Time-Matched Plasma Concentration of HBI-8000

### 12-Lead Holter Electrocardiogram Measurements

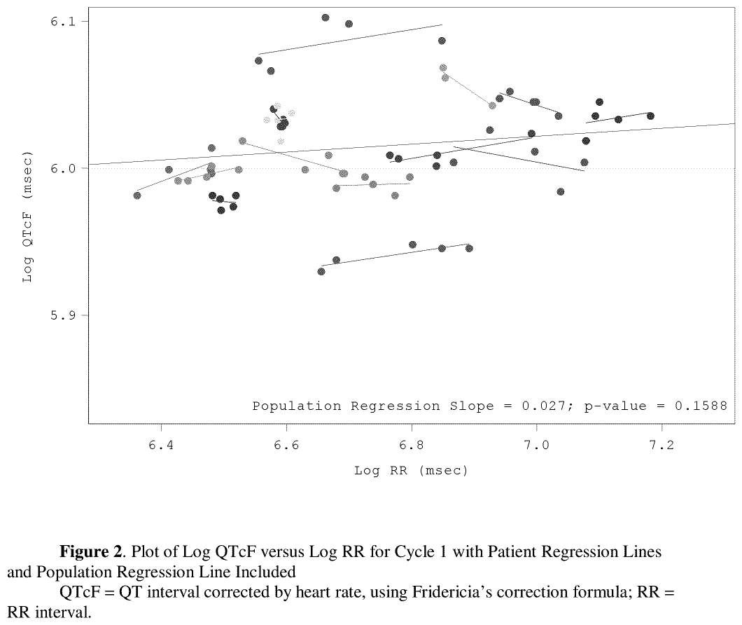

All patients in the pharmacodynamic population (N=16) had a baseline QTcF or QT interval value on Holter ECG below 450 ms. Following administration of a single dose of HBI-8000 at 20, 30, or 40 mg on C1D1, the change from baseline (A) ventricular heart rate remained essentially stable across doses evaluated over the 0 to 4 hour observation interval, indicating that the QT interval corrected by heart rate (QTcF; QTc corrected using Fridericia's correction formula) was unlikely to be impacted by changes in the heart rate. This was supported by the scatter plot of individual patient log QTcF versus log RR values, which showed no statistically significant (p=0.1588) unidirectional trends between these ECG intervals (see FIG. 2). Thus, this observation excluded the potential of heart rate as a confounding factor to the assessment of effect of plasma concentration of HBI-8000 on QTcF.

Mean and median ΔQTcF showed no discernible change across time points at the 30 mg dose (median values of 0 to −1.5 ms), but a decrease was observed at the 40 mg dose (median values of −3.0 to −11.5 ms), with the highest median decrease observed at 4 hours post dose (i.e., −11.5 ms), the last time-point evaluated near Cₘₐₓ. Two patients received the 20 mg BIW dose of HBI-8000 had a left or right bundle branch block, which precluded estimation of the QT values. Consequently, QTcF evaluation was available for only 1 of the 3 patients in the 20 mg dose cohort.

### Relationship Between HBI-8000 Concentrations/Exposure and ECG QT Interval

An inverse relationship was observed between ΔQTcF and the HBI-8000 concentrations, with QTcF decreasing with increased HBI-8000 exposure. Concentration-QT modeling indicated a negative slope between ΔQTcF and the HBI-8000 concentrations (slope estimate [95% CI] of −0.02154 [−0.03426, −0.008814]. This inverse relationship between plasma concentration and ΔQTcF revealed by concentration-QT modeling was consistent with the observed trend at the 40 mg dose, although a similar inverse relationship was not detected at the 30 mg dose. Model residual versus time examination excluded the potential of time as a confounding factor that could have influenced the statistical results, and no positive hysteresis was found.

The model predicted a mean ΔQTcF of −2.3, −2.5, and −5.6 ms at the geometric mean Cₘₐₓ for the 20, 30, and 40 mg HBI-8000 doses, respectively (Table 5), with the mean upper 90% confidence limits of −1.1 to −2.8 ms, well below the 10 ms threshold for a significant effect (Table 5).

TABLE 5
Statistical Estimates of Change from Baseline QTcF
at HBI-8000 Maximum Plasma Drug Concentration

| | | | Mean | |
|---|---|---|---|---|
| | | Geometric | Change | |
| | N Patients/ | Mean Cₘₐₓ | in QTcF | |
| Dose | n Observations | (ng/mL) | (ms) | 90% CI |
| 20 mg | 1/5 | 104.9 | −2.259 | (−3.373, −1.145) |
| 30 mg | 6/30 | 117.6 | −2.533 | (−3.782, −1.284) |
| 40 mg | 7/32 | 259.3 | −5.584 | (−8.339, −2.830) |

CI = confidence interval; Cₘₐₓ = maximum plasma drug concentration; QTcF = QT interval corrected by heart rate, using Fridericia's correction formula.
Notes:
Estimates were made using a mixed effects model for the calculated 12-Lead Holter time-matched change from baseline QTcF values, with fixed continuous effects for plasma concentration and baseline values (corrected for mean), plus patient-level random effects for intercept.
All Cycle 1 Day 1 time-points for all treatments were included in this model. Concentrations below the lower limit of quantitation were treated as a concentration of zero.

This sub-study was designed to investigate if HBI-8000 at a maximum plasma concentration would prolong QTcF. Holter ECGs were collected up to 4 hours post dose but the tmax in this study was greater than the 4 hours predicted from the previous single agent PK study of HBI-8000 in Japan. Therefore the median plasma concentrations at 4 hours were 17%, 19%, and 15% lower than the Cmax values for the 20, 30, and 40-mg doses, respectively. Because no hysteresis effect was found and because ΔQTcF showed an inverse relationship with the HBI-8000 concentration, with a greater decrease in the QTcF at higher concentrations and dose levels, this misalignment with tmax is not expected to affect the conclusion of lack of significant effect on QTcF in this dose range. In this study, no QTcF values were >450 ms and no ΔQTcF values were >30 ms. It was concluded that HBI-8000 at the doses and regimen administered did not prolong QTcF.

### Preliminary Anti-Tumor Effects

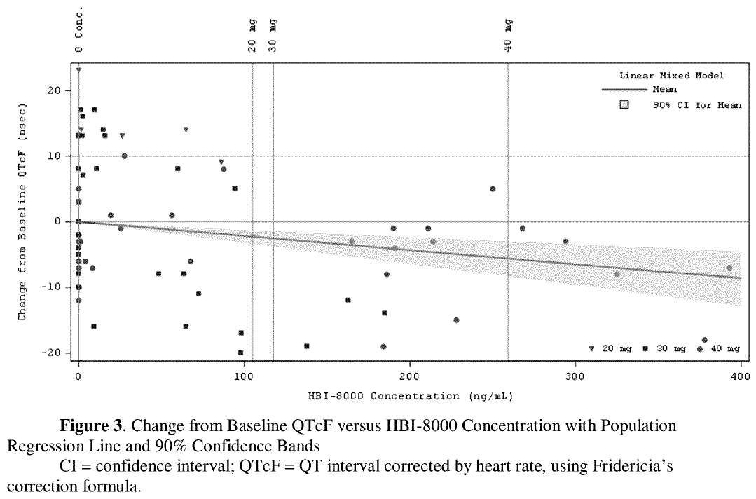

Among the 17 subjects enrolled, 15 had tumor assessments after receiving HBI-8000 and nivolumab. Signals of antitumor effects were evident across melanoma, RCC, and NSCLC (FIG. 3). When tumor response was assessed by RECIST v.1.1, all 5 melanoma patients responded with 1 complete response (CR) and 4 partial responses (PR). Of the 3 NSCLC patients, 1 had a PR, 2 with stable disease but no progressive disease. In patients with RCC, 2 of 7 had a PR, 1 stable disease, and 4 had progressive disease. Tumor responses were observed at all dose levels (FIG. 3).

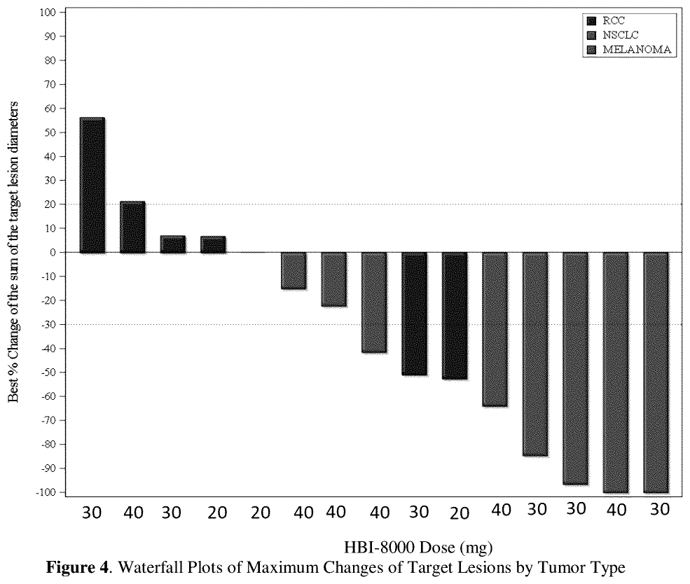

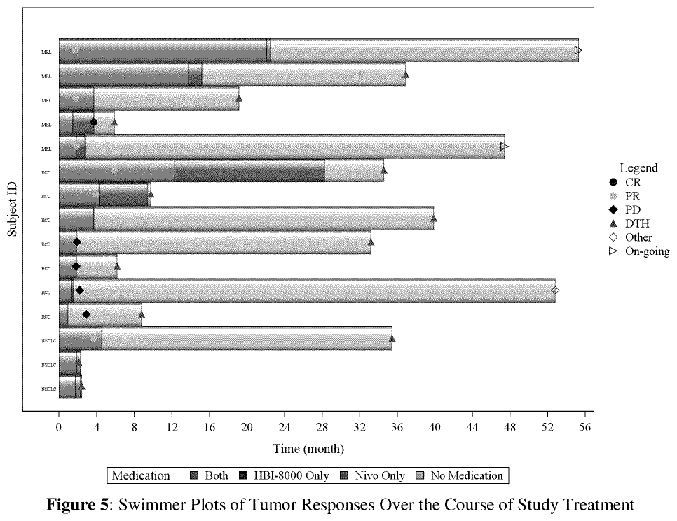

The time course of the clinical response was also analyzed (FIG. 4). The observation of anti-tumor effects started early and became clinically evident over ensuing follow-up. The earliest objective response was observed at the first scheduled tumor assessment by imaging studies after 8 weeks of treatment at the end of Cycle 2. The longest time to response was 32 months after treatment initiation in one patient, although nivolumab and HBI-8000 administration for this patient ceased after 16 months before a PR was observed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a subject with an HDAC inhibitor (HDACi), comprising administering to the subject an effective amount of an HDACi that causes no increase in QTc, QTcF, or heart rate (HR).

2. The method of claim 1, wherein said administering of HDACi causes a decrease in QTc, QTcF, or HR in the subject.

3. The method of claim 1, wherein administering the HDACi to subject at increasing dosage results in reduced QTcF.

4. The method of claim 1, wherein administering the HDACi to the subject results in no change in HR or a decrease in HR.

5. The method of any of claim 1, wherein the HDACi inhibits class I and class IIb HDACs.

6. The method of claim 5, wherein the HDACi inhibits one or more of HDAC1, HDAC2, HDAC3, or HDAC10.

7. The method of claim 6, wherein the HDACi inhibits all of HDAC1, HDAC2, HDAC3, and HDAC10.

8. The method of claim 1, wherein the HDACi is tucidinostat (chidamide).

9. The method of claim 1, wherein the effective amount is an amount effective to treat cancer.

10. The method of claim 1, wherein the cancer is an advanced solid tumor or hematological cancer.

11. The method of claim 10, wherein the cancer is one or more of melanoma, renal cell carcinoma, or non-small cell lung cancer (NSCLC).

12. The method of claim 11, wherein the effective amount is about 5 mg to about 80 mg per day.

13. The method of claim 12, wherein the HDACi is administered as an oral dose.

14. The method of claim 13, wherein the HDACi is administered at a dose of about 20 mg, about 30 mg, or about 40 mg once daily during a cycle.

15. The method of claim 14, wherein the cycle is at least about two days in duration.

16. The method of claim 15, wherein the cycle is from about one week to about ten weeks in duration.

17. The method of claim 16, further comprising administering an anti-cancer agent, PD-1 inhibitor or a PD-L1 inhibitor.

18. The method of claim 17, wherein the PD-1 inhibitor or PD-L1 inhibitor is administered on day 2 of the cycle.

19. The method of claim 18, wherein the PD-1 inhibitor is an anti-PD-1 antibody.

20. The method of claim 19, wherein the anti-PD-1 antibody is nivolumab, wherein nivolumab is administered to the subject at a dose of 240 mg per administration every two (2) weeks.
